(19) United States
(12) Patent Application Publication
(10) Pub. No.: US 2012/0059261 A1
(43) Pub. Date: Mar. 8, 2012

(54) Dual Constrained Methodology for IMT Measurement

(76) Inventor: Jasjit S. Suri, Roseville, CA (US)

(21) Appl. No.: 13/219,695

(22) Filed: Aug. 28, 2011

Related U.S. Application Data
(63) Continuation-in-part of application No. 12/799,177, filed on Apr. 20, 2010.
Application No. 12/799,177 is a continuation-in-part of application No. 12/802,431, filed on Jun. 7, 2010.
Application No. 12/802,431 is a continuation-in-part of application No. 12/896,875, filed on Oct. 2, 2010.
Application No. 12/896,875 is a continuation-in-part of application No. 12/960,491, filed on Dec. 4, 2010.
Application No. 12/960,491 is a continuation-in-part of application No. 13/053,971, filed on Mar. 22, 2011.
Application No. 13/053,971 is a continuation-in-part of application No. 13/077,631, filed on Mar. 31, 2011.
Application No. 13/077,631 is a continuation-in-part of application No. 13/107,935, filed on May 15, 2011.

(51) Int. Cl. A61B 8/00; A61B 8/14

(52) U.S. Cl. 600/440; 600/443

(57) ABSTRACT

A computer-implemented system and method for intima-media thickness (IMT) measurements using a validation embedded segmentation method. Various embodiments include receiving biomedical imaging data and patient demographic data corresponding to a current scan of a patient; checking the biomedical imaging data in real-time to determine if an artery of the patient has a atherosclerosis deposit in a proximal wall of the artery; acquiring arterial data of the patient as a combination of longitudinal B-mode or M-mode and transverse B-mode or M-mode data; using a data processor to automatically recognize the artery by embedding anatomic information; using the data processor to calibrate a region of interest around the automatically recognized artery; automatically computing the lumen intima and media-adventita borders by evolving the initial lumen intima and initial media-adventita borders constrained by distances based on polyline method or centerline method; and determining the intima-media thickness (IMT) of an arterial wall of the automatically recognized artery.

### PRIORITY APPLICATIONS

This is a continuation-in-part patent application of co-pending patent application Ser. No. 12/799,177; filed. Apr. 20, 2010 by the same applicant. This is also a continuation-in-part patent application of co-pending patent application Ser. No. 12/802,431; filed Jun. 7, 2010 by the same applicant. This is also a continuation-in-part patent application of co-pending patent application Ser. No. 12/896,875; filed Oct. 2, 2010 by the same applicant. This is also a continuation-in-part patent application of co-pending patent application Ser. No. 12/960,491; filed Dec. 4, 2010 by the same applicant. This is also a continuation-in-part patent application of co-pending patent application, Ser. No. 13/053,971 (title: IMAGING BASED SYMPTOMATIC CLASSIFICATION AND CARDIOVASCULAR STROKE RISK SCORE ESTIMATION); filed Mar. 22, 2011 by the same applicant. This is also a continuation-in-part patent application of co-pending patent application, Ser. No. 13/077,631 (title: ULTRASOUND CAROTID MEDIA WALL CLASSIFICATION AND IMT MEASUREMENT IN CURVED VESSELS USING RECURSIVE REFINEMENT AND VALIDATION); filed Mar. 31, 2011 by the same applicant. This is also a continuation-in-part patent application of co-pending patent application, Ser. No. 13/107,935 (title: ATHEROMATICT™: IMAGING BASED SYMPTOMATIC CLASSIFICATION AND CARDIOVASCULAR STROKE INDEX ESTIMATION); filed May 15, 2011 by the same applicant. This present patent application draws priority from the referenced co-pending patent applications. The entire disclosures of the referenced co-pending patent applications are considered part of the disclosure of the present application and are hereby incorporated by reference herein in its entirety.

### TECHNICAL FIELD

This patent application relates to methods and systems for use with data processing, data storage, and imaging systems, according to one embodiment, and more specifically, for ultrasound image processing.

### BACKGROUND

The state of Atherosclerosis in carotids or other blood vessels can be studied using MRI or Ultrasound. Because ultrasound offers several advantages like real time scanning of carotids, compact in size, low cost, easy to transport (portability), easy availability and visualization of the arteries are possible, Atherosclerosis quantification is taking a new dimension using ultrasound. Because one can achieve compound and harmonic imaging which generates high quality images with ultrasound, it is thus possible to do two-dimensional (2D) and three-dimensional (3D) imaging of carotid ultrasound for monitoring of Atherosclerosis.

In recent years, the possibility of adopting a composite thickness of the tunica intima and media, i.e., an intima-media thickness (hereinafter referred to as an “IMT”) of carotid arteries, as surrogate marker for cardiovascular risk and stroke. Conventional methods of imaging a carotid artery using an ultrasound system, and measuring the IMT using an ultrasonic image for the purpose of diagnosis are being developed.

A conventional measuring apparatus can measure an intima-media thickness of a blood vessel using an ultrasound device to scan the blood vessel. Then, for example, an image of a section of the blood vessel including sections of the intima, media and adventitia is obtained. The ultrasound device further produces digital image data representing this image, and outputs the digital image data to a data analyzing device.

The intima, media and adventitia can be discriminated on the basis of changes in density of tissue thereof. A change in density of tissue of the blood vessel appears as a change of luminance values in the digital image data. The data analyzing device detects and calculates the intima-media thickness on the basis of the changes of luminance values in the digital image data. The digital image data can include a plurality of luminance values each corresponding to respective one of a plurality of pixels of the image. The data analyzing device can set a base position between a center of the blood vessel and a position in a vicinity of an inner intimal wall of the blood vessel on the image, on the basis of a moving average of the luminance values. The data analyzing device can detect a maximum value and a minimum value from among the luminance values respectively corresponding to a predetermined number of the pixels arranged from the base position toward a position of an outer adventitial wall on the image. The data analyzing device can then calculate the intima-media thickness on the basis of the maximum value and the minimum value.

The major challenges which can be affected in finding the IMT are: (a) how well the ultrasound probe is gripped with the neck of a patient to scan the carotids; (b) how well the ultrasound gel is being applied; (c) the orientation of the probe; (d) demographics of the patient; (e) skills of the sonographer or vascular surgeon; (f) gaps in the intensity distribution along the adventitia walls of the carotid ultrasound images; (g) shadows cones in the adventitia borders due the presence of calcium deposits; (h) threshold chosen for finding the peaks corresponding to the LI and MA points for each signal orthogonal to the lumen; (i) variability in the lumen region; (j) variability in the geometric shapes of the carotid scans such as convex, concave, up-hill, down-hill, and finally, (k) handing the large databases to process large number of images.

Thus, a system and method for fast, reliable and automated method for IMT measurements is needed.

The application technique (called CMUDS) is being benchmarked with the previous published system, CALEX in the publication: J Ultras Med 2010a, 29:399-418. Here on when we use the word CALEX, it means it is being referred to the publication: J Ultras Med 2010a, 29:399-418.

### BRIEF DESCRIPTION OF THE DRAWINGS

FIG. 1 shows the IMT measurement system.

FIG. 2 is the overall AtheroEdge™ system.

FIG. 3 shows the image reduction processor based on wavelet transform or pyramid filter, which can down sample the image.

FIG. 4 shows the process for despecking filtering. The process uses the scaling of the original pixel. The noise variance process is being used by the scale processor.

FIG. 5 is the filter processor.

FIG. 6 shows the computation of the noise variance processor.

FIG. 7 shows a cascaded two stage processor.

FIG. 8 cascaded recognition processor and validation processor.

FIG. 9 shows the artery recognition processor.

FIG. 10 is the details of the artery validation processor.

FIG. 11 is the lumen identification system.

FIG. 12 shows the Lumen Classifier Processor for segmentation of Lumen region in the longitudinal carotid image.

FIG. 13 is the Calibration Processor (stage II of the IMT system).

FIGS. 14 (a), 14 (b), 14 (c) and 14 (d) shows the external energy term images.

FIGS. 15 (a), 15 (b), 15 (c) and 15 (d) shows the LI and MA initialization.

FIG. 16 (a) shows the input and FIG. 16 (b) results of the CMUDS.

FIG. 16 (c), FIG. 16 (e), FIG. 16 (g), FIG. 16 (i) show the LIMA initializations.

FIG. 16 (d), FIG. 16 (f), FIG. 16 (h) and FIG. 16 (j) are the CMUDS results.

FIG. 17 (a) shows the CMUDS LIMA border in comparison Reader-1 LIMA.

FIG. 17 (b) shows the Zoomed version of the region of interest taken from FIG. 17 (a). FIG. 17 (h) also shows the LIMA from CMUDS vs. LIMA from Reader-1.

FIG. 17 (c) and FIG. 17 (d) shows the LIMA profiles of CMUDS vs. LIMA profiles of Reader-1.

FIG. 17 (d) and FIG. 17 (e) shows the LIMA profiles of CMUDS vs. LIMA profiles of Reader 1.

FIG. 17 (e) and FIG. 17 (f) shows the LIMA profiles of CMUDS vs. LIMA profiles of Reader-1.

FIG. 18 (a) are the LIMA profiles by relaxing the CMUDS's constraints.

FIG. 18 (b) are the LIMA profiles by enforcing the constraints of CM LIDS.

FIG. 18 (c) and FIG. 18 (d) are LIMA profiles for example patient without constraints and with constraints in CMUDS, respectively.

FIG. 18 (e) and FIG. 18 (f) are LIMA profiles for another example patient without constraints and with constraints in CMUDS, respectively.

FIG. 18 (g) and FIG. 18 (h) are LIMA profiles for another example patient without constraints and with constraints in CMUDS, respectively.

FIG. 18 (i) and FIG. 18 (j) are LIMA profiles for another example patient without constraints and with constraints in CMUDS, respectively.

FIG. 19 (a) is the Bland-Altman Plots between CUMDS and Reader-1.

FIG. 19 (b) is the Bland-Altman Plots between CUMDS and Reader-2.

FIG. 19 (c) is the Bland-Altman Plots between CALEX and Reader-1.

FIG. 19 (d) is the Bland-Altman Plots between CALEX and Reader-2.

FIG. 20 is the table showing the parameters for estimation of ADF borders (stage I).

FIG. 21 is the table showing the parameters used for the CUMDS (calibration stage).

FIG. 22 show the comparison of mean IMT values between CMUDS, CALEX, Reader-1 and Reader-2.

FIG. 23 show the comparison of performance error values between CALEX and CMUDS.

FIG. 24 is a processing flow diagram illustrating an example embodiment of a computer-implemented system and method for fast, reliable, and automated embodiments for using a validation embedded multi-resolution edge flow approach to vascular ultrasound for intima-media thickness (IMT) measurement as described herein.

FIG. 25 shows a diagrammatic representation of machine in the example form of a computer system within which a set of instructions when executed may cause the machine to perform any one or more of the methodologies discussed herein.

### DETAILED DESCRIPTION

Recognition of the carotid artery consists of finding a regional layer close to the carotid artery and possibly all along the carotid artery in the image frame. This recognition process must ensure that we are able to distinguish the carotid artery layer from other veins such as jugular vein (JV). We modeled the carotid artery recognition process by taking the hypothesis that carotid artery's far wall adventitia is the brightest in the ultrasound scan frame; hence if we can automatically find this layer, then segmentation process of the far wall would be more systematic and channeled. Since the scanning process of carotid artery yields varying geometries of the carotid artery in the ultrasound scans, one has to ensure that the recognition process is able to handle various geometric shapes of the carotid arteries in the images. The process of location of far adventitia bright layer in the image frame can be supported by the fact that it is very close to lumen region, which carries the blood to the brain. Taking these two properties of the carotid artery ultrasound scan, this patent application has modeled the recognition process as a tubular model where the walls are considered as bright layers of the scan which can be picked up by the high intensity edge detector. Our edge model must keep in mind that the far adventitia layers are about a millimeter thick (which is about 16 pixels in image frame). Thus one would need to find an edge operator (preferably Gaussian in nature) which has an ability to have a width (scale) region of as wide as 8 pixels in the image frame. We have modeled this width to be the scale factor of the Gaussian kernel, where the scale is the standard deviation of the edge operator. The ability of finding this edge can be obtained by convolving the image region with a derivative of the Gaussian Kernel having a scale factor as rationalized in the edge model. Thus the whole idea of finding automatically the far adventitia border can be brought in the frame work of scale-space, where the image is convolved with first or higher order derivatives of Gaussian Kernel with known scale (s). While the scale-space model is fancy in itself, one must remember that it is very important to have the scale nearly fitting the far adventitia border region. Since the image frame is large enough to have a wider scale, we therefore have further adapted an approach where the scale-space model will behave consistent with respect to the image size. This requires that image be down sampled to half before the scale-space model can be adapted. Thus one can call this framework to be more like a multi-resolution thereby using the correct scale for capturing the edges of the far adventitia layers. Thus our architecture for stage I is the recognition of the far adventitia location in the grayscale image of the carotid artery using multi-resolution approach in scale-space framework.

In the following description, for purposes of explanation, numerous specific details are set forth in order to provide a thorough understanding of the various embodiments. It will be evident, however, to one of ordinary skill in the art that the various embodiments may be practiced without these specific details.

This patent application discloses various embodiments of a computer-implemented system and method for fast, reliable, and automated embodiments for vascular ultrasound for validation embedded LIMA segmentation and intima-media thickness (IMT) measurement. In particular, this patent application discloses various embodiments of a computer-implemented system and method for intima-media thickness (IMT) measurements using (a) a validation embedded segmentation method in stage I, while stage-II consists of (b) automated initialization of LI and MA borders and (c) constrained snake evolution using constraints, (d) LI/MA refinement. The various embodiments described herein also include the features described in more detail below.

Coarse to Fine Resolution Processing: Previous art has focused on methods for either classification of media layer or finding the MA edges in the manual designated Region of Interest (ROI). Since it is manual ROI, it is time consuming and non-practical for clinical applications, we have developed a new method which is fast, accurate, reliable and very practical for IMT measurement for carotids, brachial, femoral and aortic blood vessels. Since the manual methods are time consuming and requires a lot of training, this applications is a two step stage process: (a) automated validation embedded artery recognition and (h) automated calibration using (i) automated LIMA initialization, (ii) constrained snake evolution and (iii) LI/MA refinement. The automated recognition process is challenging given the Jugular vein in the neighborhood. Our concept is to recognize the artery in a smaller image with a high speed (so-called coarse resolution) and recognize the artery. The spotted artery can then be seen in the fine resolution or high resolution. This will allow processing the pixels in the correct region of interest. The statistics of the neighboring pixels will not affect the region of interest, which is where the accurate LIMA borders need to be determined. Normally, arteries are about 10 mm wide while the media thickness is about 1 mm wide. It is also known from our experience that the image resolution is about 15-17 pixel per mm. If we can bring the original resolution to a coarse resolution by down sampling one step, we can bring the media layer to about 8 pixels per mm. Further, if this coarse resolution is down sampled by another half, then one can bring the image resolution from 8 pixels/mm to 4 pixels/mm. Thus, if the coarse resolution of the arterial ultrasound vessels has a medial thickness of 4 pixels/mm, one can easily detect such edges by convolving the higher order derivatives of Gaussian kernel with the coarse resolution image. Thus, a new concept here (in stage I) is to automatically detect the arterial wall edges by down sampling the image and convolving the coarse images to higher order derivatives of Gaussian kernels. This allows the media layer to be automatically determined. Such an approach for automated media layer detection from fine to coarse resolution will further improve the region of interest determination. The art of changing the fine to coarse resolution has been popular in computer vision sciences. There are several methods available to converting the image from high resolution to coarse resolution. One of them is wavelet-based method where wavelets are being applied for down sampling the image to half. Another method can be hierarchical down sampling method using Peter Burt's algorithm. Thus the first advantage of the current system is automated recognition of the artery at coarse resolution and then using the MA border for visualization and recognition at the fine resolution (up-sampled resolution). This scheme has several advantages to it:
(1) Robustness and Accurate Wall Capture: it is very robust because the higher order derivative kernels are very good in capturing the vessel walls (See, A Review on MR Vascular Image Processing Algorithms: Acquisition and Pre-filtering: Part I Suri et. al., IEEE TRANSACTIONS ON INFORMATION TECHNOLOGY IN BIOMEDICINE, VOL. 6; NO. 4, pp. 324-337, DECEMBER 2002; and A Review on MR Vascular Image Processing: Skeleton Versus Nonskeleton Approaches: Part II, Suri et al., IEEE TRANSACTIONS ON INFORMATION TECHNOLOGY IN BIOMEDICINE, VOL. 6, NO. 4, DECEMBER 2002).
(2) Scale-space Method for Automated Recognition: Since the scale of the derivative Gaussian Kernels are determined using the anatomic information of the artery along with the multi-resolution framework, the system is robust to find the far adventitia (ADP) borders.
(3) Validation Embedded Segmentation of Vascular IMT estimation: Here the recognition of artery has been validated by the anatomic information during the segmentation process. Since lumen is the anatomic information which is blood carrier to brain and is next to the far adventitia borders, which needs to be located, therefore, this patent application uses the anatomic information (lumen) to ensure that the far adventitia borders (ADF) are robustly computed along the CCA/ICA, and do not penetrate the lumen region or near wall region. This adds robustness to our automated recognition and IMT measurement system.
(4) Easter than the conventional processing: Since the recognition is strategized at coarse level down sampled once or twice from its original size of the image, it is therefore processing ¼^{th} the number of pixels for automated recognition of the media layer. This improves the speed of the system.
(5) Independent of Orientation of the vascular scan: Another major advantage to the system is that these Gaussian kernels are independent of the orientation of the blood vessels in the image. Since the ultrasound vascular scans do not always have the vessel orientation horizontal with respect bottom edge of the image, manual methods can pose a further challenge towards the region of interest estimation.
(6) Guiding Method for the calibration System: Since the recognition is followed by the calibration process, the calibration system becomes very robust since the calibration processing is done in the region of interest determined by the automated validation embedded recognition system. Thus the calibration system adds the value determined by the automated recognition system. This is applicable for blood vessels such as: carotid, femoral, aortic and brachial. Such a combination where the calibration system is guided by the automated recognition system helps in mass processing of huge databases.
(7) Running the Mass IMT system for Clinical Analysis: Since the recognition is automated followed by the calibration sub-system, the largest value such a system would deliver will be in its real time usage for analysis of IMT measurement on a large databases. Running clinical databases on still images would be even more beneficial because such a system would be completely automated in terms of arterial recognition and IMT measurement.
(8) Applications: Since the ultrasound probes use almost the same frequency of operation for scanning the vascular arteries such as carotid, femoral, brachial and aortic, it is thus possible to use such a cascaded system for these blood vessels.

In the prior art, we have seen that the speckle reduction has been used for removing speckles in the ultrasound images. Though speckle reduction is common in ultrasound imaging, but the way speckle reduction is used here is very conservative. The idea here is to find out where the LIMA borders are using automated recognition system and then apply the local statistical speckle reduction filter in specific set of pixels which come under the LIMA band or media layer. Such a strategy allows multiple advantages:
(1) Avoiding Large Computation Times on Speckle Reduction: The computation time for speckle reduction is not wasted in such a strategy, unlike conventional methods, where the speckle reduction is part of the whole streamline flow and is being run on the whole image.
(2) Speckle Reduction is implemented on the original raw intensity in the region estimated at a Coarse Resolution: Second, the speckle reduction filter is run in the automated recognized region which is actually applied on the original image rather than on the coarse image. This way the original speckles are, removed preserving the intensities of high gradient structures like LI and MA peaks. This is very important because the calibration system acts on these speckle reduction region of interest.
(3) Guidance to the calibration System: The calibration system is guided by the speckle reduction filter which is optimized for the region of interest.

Extracting LIMA borders in presence of Calcium Shadow: Calcium is an important component of the media layer. It is not exactly known how the calcium is formed, but it is said that calcium accumulates in the plaques. During the beginning of Atherosclerosis disease, the arterial wall creates a chemical signal that causes a certain type of WBC (white blood cells) such as monocytes and T cells that attaches the arterial wall. These cells then move into the wall of the artery. These T cells or monocycles are then transformed into foam cells, which collect cholesterol and other fatty materials and trigger the growth of the muscle cells (which are smooth in nature) in the artery. Over time, it is these fat-laden foam cells that accumulate into plaque covered with a fibrous cap. Over time, the calcium accumulates in the plaque. Often times, the calcium is seen in the near wall (proximal wall) of the carotid artery or aortic arteries. This causes the shadow cone formation in the distal wall (far wall). As a result the LI boundaries are over computed from its actual layer. The shadow causes the LI lining over the actual LI boundary. As a result, the LI-MA distances are over computed in the shadow zone. Because of this, the IMT formation is over computed in these cases.

This application particularly takes care of IMT computation during the shadow cone formation. We will see how the actual LI boundaries are recovered if calcium is present causing the shadow cone. As a result, the IMT computation has the following advantages when using shadow cones.
(1) Accurate IMT computation in real time when the calcium is present in the proximal wall (near wall) causing the shadow cone formation.
(2) The system allows computing the IMT in both cases: (a) when calcium is present and when calcium is not present.

### BRIEF SUMMARY OF AN EXAMPLE EMBODIMENT

The completely automated technique we developed and named CMUDS (class of AtheroEdge™ systems) consists of two steps: (i) the automated validation embedded recognition of the CA in the image frame, and (ii) the segmentation of the far carotid artery wall using (i) automated initialization of LIMA borders, (ii) constrained LIMA evolution and (iii) LI/MA refinement. The output of the stage II yields LI/MA profiles which are then used for the IMT measurement.

Cropping System: Preliminarily, the raw ultrasound image is automatically cropped in order to discard the surrounding black frame containing device headers and image/patient data (1). If the image came in DICOM format, we relied on the data contained in the specific field named SequenceOfUltrasoundRegions, which contains four sub-fields that mark the location of the image containing the ultrasound representation. These fields are named RegionLocation (their specific label is xmin, xmax, ymin and ymax) and they mark the horizontal and vertical extension of the image. The raw B-Mode image is then cropped in order to extract only the portion that contains the carotid morphology. Those skilled in the art of DICOM will know that if the image came in from other formats or if the DICOM tags were not fully formatted, one can adopt a gradient-based procedure. We computed the horizontal and vertical Sobel gradient of the image. The gradients repeat similar features for the entire rows/columns without the ultrasound data: they are zero at the beginning and at the end. Hence, the beginning of the image region containing the ultrasound data can be calculated as the first row/column with gradient different from zero. Similarly, the end of the ultrasound region is computed as the last non-zero row/column of the gradient.

Automatic Recognition of the CA: To automatically identify the CA in the image frame, we developed a novel and low-complexity procedure. Following sample steps are used for automatic CA recognition, starting with the automatically cropped image which constitutes the input of the procedure.
Downsampling. The image was first down-sampled by a factor of 2 (i.e.: the number of rows and columns of the image was halved).
Speckle reduction. Speckle noise was attenuated by using a first-order statistics filter (named as km by the authors (2, 3)), which gave the best performance in the specific case of carotid imaging. This filter is defined by the following equation:

J_{x,y}=Ī+k_{x,y}(I_{x,y}−Ī) (1)
where, I_{x,y} is the intensity of the noisy pixel, Ī is the mean intensity of a N×M pixel neighborhood and k_{x,y} is a local statistic measure. The noise-free pixel is indicated by J_{x,y}. Loizou et al. (2) mathematically defined

$${k}_{x , y} = \frac{{σ}_{I}^{2}}{{\overline{I}}^{2} {σ}_{I}^{2} + {σ}_{n}^{2}} ,$$

where σ₁² represents the variance of the pixels in the neighborhood, and σₙ² the variance of the noise in the cropped image. An optimal neighborhood size was shown to be 7×7.
Higher order Gaussian derivative filter. The despeckled image was filtered by using a first order derivative of a Gaussian kernel filter. It is possible to observe how the CA walls become enhanced to white. The sigma parameter of the Gaussian derivative kernel was taken equal to 8 pixels, i.e. to the expected dimension of the WIT value. In fact, an average IMT value of say 1 mm corresponds to about 16 pixels in the original image scale and, consequently, to 8 pixels in the down-sampled scale.
AD_{F} refinement using anatomic information and spike removal. The traced AD_{F} profile could be characterized by spikes and false points identification. This could be due to several reasons such as variations in intensities, gaps in the media walls, presence of jugular vein, shadow effects or combination of these. This innovation, therefore introduced a validation protocol, which provides a check on the far adventitia (AD_{F}) profile ensuring that the location of carotid artery is at correct place and the segmentation edge is not very bumpy. This validation step refines the far adventitia (AD_{F}) profile and is done in two steps: (a) refinement using anatomic lumen and (b) spike removal.
   Refinement by anatomic reference (Lumen). This check has been introduced to avoid error conditions of AD_{F} curve protruding into the lumen vessel. Thus, the refinement step requires the identification of the lumen region automatically. We have modeled the lumen segmentation region as a classification process with two classes. Carotid characteristics can be thought of as a mixture model with varying intensity distributions. This is because (a) the pixels belonging to the vessel lumen are characterized by low mean intensity and low standard deviation; (b) pixels belonging to the adventitia layer of the carotid wall are characterized by high mean intensity and low standard deviation; and (c) all remaining pixels should have high mean intensity and high standard deviation. As a result of this distribution, we derived a bi-dimensional histogram (2DH) of the carotid image. For each pixel, we considered a 10×10 neighborhood of which we calculated the mean value and the standard deviation. The mean values and the standard deviations were normalized to 0 and 1 and were grouped into 50 classes each having an interval of 0.02. The 2DH was then a joint representation of the mean value and standard deviation of each pixel neighborhood. It is well established that pixels belonging to the lumen of the artery are usually classified into the first classes of this 2DH: expert sonographer manually traced the boundaries of the CCA lumen and observed the distribution of the lumen pixels on the 2DH. Overall results revealed that pixels of the lumen have a mean values classified in the first 4 classes and a standard deviation in the first 7 classes. We therefore consider a pixel as possibly belonging to the artery lumen if its neighborhood intensity is lower than 0.08 and if its neighborhood standard deviation is lower than 0.14. This method shows how the local statistic is effective in detecting image pixels that can be considered as belonging to the CCA lumen. The AD_{F} points along the CA are considered one by one. For each AD_{F} point:
   1. We consider the sequence of the 30 pixels above it (i.e., the 30 pixels located above the AD_{F} point, towards the top of the image, and, therefore, with lower row indexes).
   2. The AD_{F} point failed the lumen test, if the AD_{F} point crosses the lumen region and has penetrated the lumen region by at least 30 pixels inside the lumen or more. These failed points must not belong to the AD_{F} boundary. These AD_{F} points which fail the lumen test are tagged as 0, while rest of the points are tagged as 1. All the AD_{F} points that tagged as 0 are deleted from the AD_{F} list.
   3. The procedure is repeated for each AD_{F} point along the CA artery.
      Those skilled in the art can use an existing method for lumen computation such as threshold methods, adaptive threshold methods or edge detection methods.

Note that even though, the lumen anatomic information, which acts as a reference, provides a good test for catching a series of wrongly computed ADF boundary, it might slip from sudden bumps which may be due to the changes in grayscale intensity due presence of unusual high intensity in lumen region or a calcium deposit in the near wall causing a shadow in far wall region. This sudden spike can then be easily detected ahead using the spike detection method.
Spike detection and removal. We implemented an intelligent strategy for spike detection and removal. Basically, we compute the first order derivative of the AD_{F} profile and check for values higher than 15 pixels. This value was chosen empirically by considering the image resolution. When working with images having approximate resolution of about 0.06 mm/pixel, an IMT value of 1 mm would be about 17 pixels. Therefore, a jump in the AD_{F} profile of the same order of magnitude of the IMT value is clearly a spike and error condition. If the spike is at the very beginning of the image (first 10 columns) or at the end (last 10 columns), then the spiky point is simply deleted. Otherwise, all spikes are considered and either substituted by a neighborhood moving average or removed.
Far adventitia AD_{F} automated tracing. Intensity profile for one column of the filtered image is then taken. The near and far walls clearly have intensity maxima saturated to the maximum value of 255. To automatically trace the profile of the far wall, we used a heuristic search applied to the intensity profile of each column. Starting from the bottom of the image (i.e. from the pixel with the higher row index), we searched for the first white region of at least 6 pixels of width. The deepest point of this region (i.e. the pixel with the higher row index) marked the position of the far adventitia (AD_{F}) layer on that column. The sequence of all the points of the columns constituted the overall AD_{F} automatically generated tracing.

Up-sampling to Fine Resolution. The AD_{F} profile is then up-sampled to the original scale and overlaid to the original image. At this stage, the carotid artery far wall is automatically located in the image frame and automated segmentation is made possible.

LI/MA Border Segmentation (stage II)

The goal for this stage in the segmentation process is the extraction of the LI and MA borders which lie in between the computed AD_{F} border and the lumen region. This region of the image in which the LI and MA borders can be found is called the guidance zone, which is empirically computed from the knowledge database.

### DETAILED DESCRIPTION OF AN EXAMPLE EMBODIMENT

In the following description, for purposes of explanation, numerous specific details are set forth in order to provide a thorough understanding of the various embodiments. It will be evident, however, to one of ordinary skill in the art that the various embodiments may be practiced without these specific details.

This patent application discloses a computer-based system and method for intima-media thickness (IMT) measurements in presence of calcium or absence of calcium in near (proximal) end of the arterial value. The embodiment is being designed for carotid, femoral, brachial and aortic arteries. IMT measurement is a very important risk marker of the Atherosclerosis disease. Typically, there are two ways to measure the arterial IMT's: (a) invasive methods and (b) non-invasive methods. In invasive methods, traditionally, intravascular ultrasound (NUS) is used for measuring vessel wall thickness and plaque deposits where special catheters are inserted in the arteries to image them. Conventional ultrasound is used for measuring IMT non-invasively, such as from carotid, brachial, femoral and aortic arteries. The main advantages of non-invasive methods are: (i) low cost; (ii) convenience and comfort of the patient being examined; (iii) lack of need for any intravenous (IV) insertions or other body invasive methods (usually), and (iv) lack of any X-ray radiation; Ultrasound can be used repeatedly, over years, without compromising the patient's short or long term health status. Though conventional methods are generally suitable, conventional methods have certain problems related to accuracy and reliability.

The IMTs are normally 1 mm in thickness, which nearly corresponds to 1 pixels on the screen or display. IMT estimation having a value close to 1 mm is a very challenging task in ultrasound images due to large number of variabilities such as: poor contrast, orientation of the vessels, varying thickness, sudden fading of the contrast due to change in tissue density, presence of various plaque components in the intima wall such as lipids, calcium, hemorrhage, etc. Under normal resolutions, a 1 mm thick media thickness is difficult to estimate using stand-alone image processing techniques. Over and above, the image processing algorithms face an even tighter challenge due to the presence of speckle distribution. The speckle distribution is different in nature from these interfaces. This is because of the structural information change between intima, media and adventitia layers of the vessel wall. As a result, the sound reflection from different cellular structures is different. The variability in tissue structure—all that happens in 1 mm of the vessel wall—brings fuzziness in the intensity distribution of the vessel wall. Under histology, media and adventitia walls are clearly visible and one can observe even their thicknesses. This 1 mm zone is hard to discern in a normal resolution image of 256×256 pixels in a region of interest (ROI) or in a higher resolution image of 512×512 pixels in a region of interest (ROI). One needs a high resolution image to process and identify the intensity gradient change in ultrasound images from lumen to intima and media to adventitia layers. The ultrasound image resolution may not be strong enough like MRI or computerized axial tomography (CAT or CT) images, which can be meaningful for soft tissue structural information display.

There are two ways to process and identify the intensity gradient change in ultrasound images from lumen to intima (LI) and media to adventitia (MA) layers: (a) have a vascular surgeon draw the LI/MA borders and compute the IMT image interactively, OR (b) have a computer determine the LI and MA borders along with IMT's. Case (a) is very subjective and introduces variability in the IMT estimation. IMT screenings are really part of the regular check-up for patients and millions of scans are done each day around the world. The manual handling of such a repetitive work flow of IMT screenings is tedious, error-prone and subject to lot of variability. Case (b) is difficult to implement, because it is difficult to identify the LI and MA borders with heavy speckle distribution and the inability of ultrasound physics to generate a clear image where the semi-automated or automated image processing methods are used for IMT estimation. Besides that, the calcium deposit in the near walls causes the shadow.

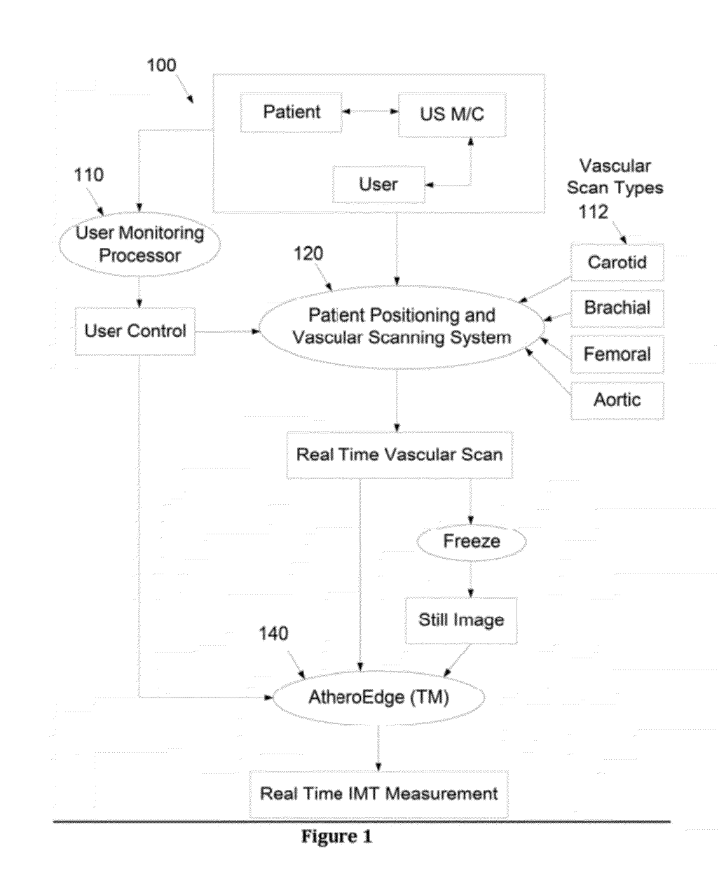

FIG. 1 shows how the main system uses AtheroEdge™ processor. The system shows how the ultrasound vascular image is acquired using the longitudinal B-mode process (block 120). The input to the system also shows that this process can take any of the four arteries: carotid, brachial, femoral and arotic (block 112). The system has ability to freeze the image as a still image, on which the IMT will be computed. User continuously monitors the process at all stages during the operation (block 110). User has control on the AtheroEdge™ software system, ultrasound machine, ultrasound probe, patient and the graphical user interface (block 110). The still image can be saved on the hard drive or CD drive. The still image can then also be transferred to an independent computer and AtheroEdge™ can be run on that system as well. At the same time AtheroEdge™ can run real time while the patient is in the vascular screening room.

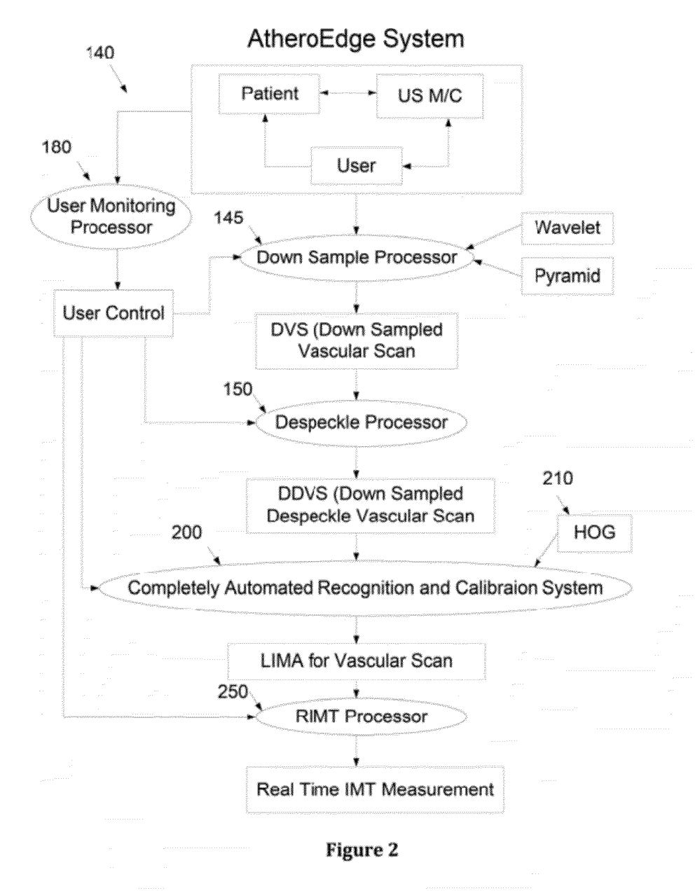

FIG. 2 shows the AtheroEdge™ system where the main components of the system are: (a) Multi-resolution Image Processor (block 145); (b) De-speckle Processor (block 150); (c) Recognition, Validation Processor and Calibration Processor (block 120 using higher order Gaussian derivative operators (HOG) block 210) and (d) RIMT Processor (block 250).

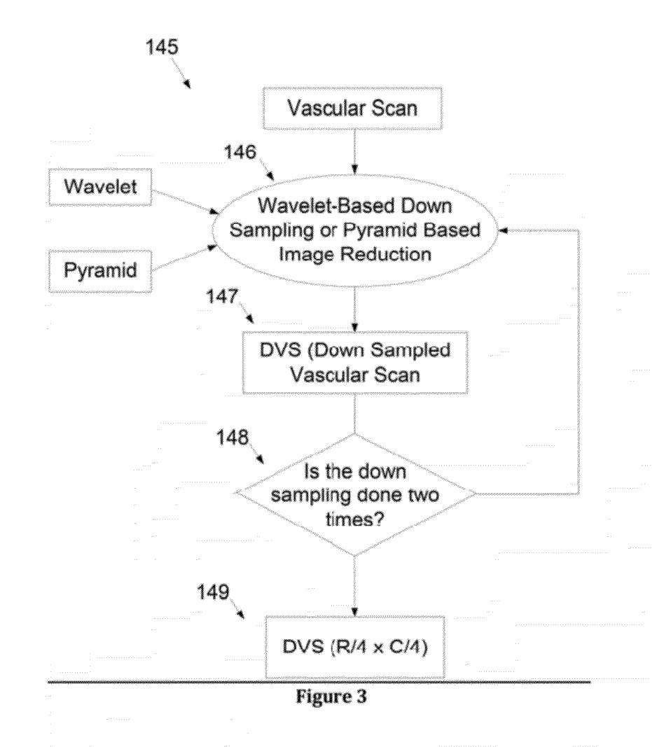

Multi-resolution image processing yields the DSVS (down sampled vascular scan) image. FIG. 3 shows the down sampling or fine to coarse resolution system (block 145). One of the four systems can be used for fine to coarse sampling. The role of the multi-resolution process is to convert the image from fine resolution to coarse resolution. Those skilled in the art of down sampling any use off the shelf down sampling methods. One of the very good down samplers are Lanczos interpolation. This is based on the sine function which can be given mathematically as

$$sin c ( x ) = \frac{sin ( π x )}{π x} .$$

Since the sine function never goes to zero, practical filter can be implemented by taking the sine function and multiplying it by a “window”, such as Hamming and Hann, giving an overall filter with finite size. We can define the Lanczos window as a sine function scaled to be wider, and truncated to zero outside of the main lobe. Therefore, Lanczos filter is a sine function multiplied by a Lanczos window. Three lobed Lanczos filter can be defined as

$$Lanczos 3 ( x ) = \{ \begin{matrix}\frac{sin ( π x ) sin ( π \frac{x}{3} )}{π x \cdot π \frac{x}{3}} , & if \vert x \vert \leq 3 \\ 0 , & if \vert x \vert > 3\end{matrix}$$

Although Lanczos interpolation is slower than other approaches, it can obtain the best interpolation results because Lanczos method attempts to reconstruct the image by using a series of overlapping sine waves to produce what's called a “best fit” curve. Those skilled in the art of down sample can also use Wavelet transform filters as they are very useful for multi-resolution analysis. The orthogonal wavelet transform of a signal f can be formulated by

$$f ( t ) = {\sum }_{k ∈ z} {c}_{j} ( k ) {ϕ}_{J , k} ( t ) + {\sum }_{J = 1}^{J} {\sum }_{k ∈ Z} {d}_{j} ( k ) {ϕ}_{j , k} ( t )$$

where the cⱼ(k) is the expansion coefficients and the dⱼ(k) is the wavelet coefficients. The basis function φ_{j,k}(t) can be presented as

φ_{j,k}(t)=2^{-j/2}φ(2^{-j}y−k),

where k, j are translation and dilation of a wavelet function φ(t). Therefore, wavelet transforms can provide a smooth approximation of f(t) at scale J and a wavelet decomposition at per scales. For 2-D images, orthogonal wavelet transforms will decompose the original image into 4 different sub-band (LL, LH, HL and HH) (block 149).

Bicubic interpolation can also be used as it will estimates the value at a given point in the destination image by an average of 16 pixels surrounding the closest corresponding pixel in the source image. Given a point (x,y) in the destination image and the point (l,k) (the definitions of l and k are same as the bilinear method) in the source image, the formulae of bicubic interpolation is

$$f ( x , y ) = {\sum }_{m = l - 1}^{l + 2} {\sum }_{n = k - 1}^{k + 2} g ( m , n ) \cdot r ( m - l - dx ) \cdot ( dy - n + k ) ,$$

Where the calculation of dx and dy are same as the bilinear method. The cubic weighting function r(x) is defined as

$$r ( x ) = \frac{1}{6} [ {p ( x + 2 )}^{3} - 4 {p ( x + 1 )}^{3} + 6 {p ( x )}^{3} - 4 {p ( x - 1 )}^{3} ] ,$$

where p(x) is

$$p ( x ) = \{ \begin{matrix}x & x > 0 \\ 0 & x \leq 0\end{matrix}$$

Bicubic approach can achieve a better performance than the bilinear method because more neighboring points are included to calculate the interpolation value.

Bilinear interpolator can also be used as it is very simple to implement. Mathematically, it is given as: if g represents a source image and f represents a destination image, given a point (x,y) in f, the bilinear method can be presented as:

$$f ( x , y ) = ( 1 - dx ) \cdot ( 1 - dy ) \cdot g ( l , k ) + dx \cdot ( 1 - dy ) \cdot g ( l + 1 , k ) + ( 1 - dx ) \cdot dy \cdot g ( l , k + 1 ) + dx \cdot dy \cdot g ( l + 1 , k + 1 ) ,$$

where l=└x┘ and k=└y┘, and the dx, dy are defined as dx=x−1 and dy=y−k respectively. Bilinear interpolation is simple. However it can cause a small decrease in resolution and blurring because of the averaging nature.

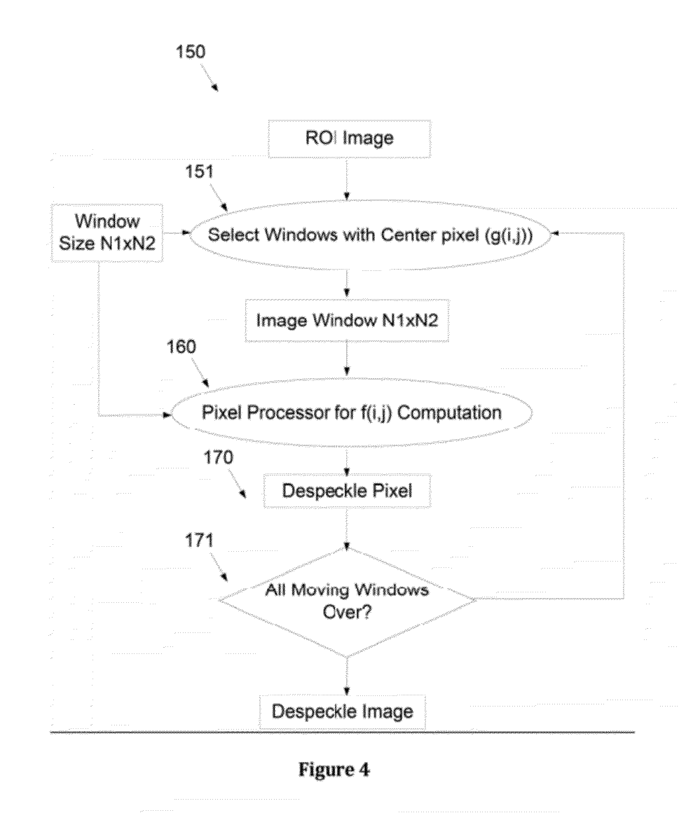

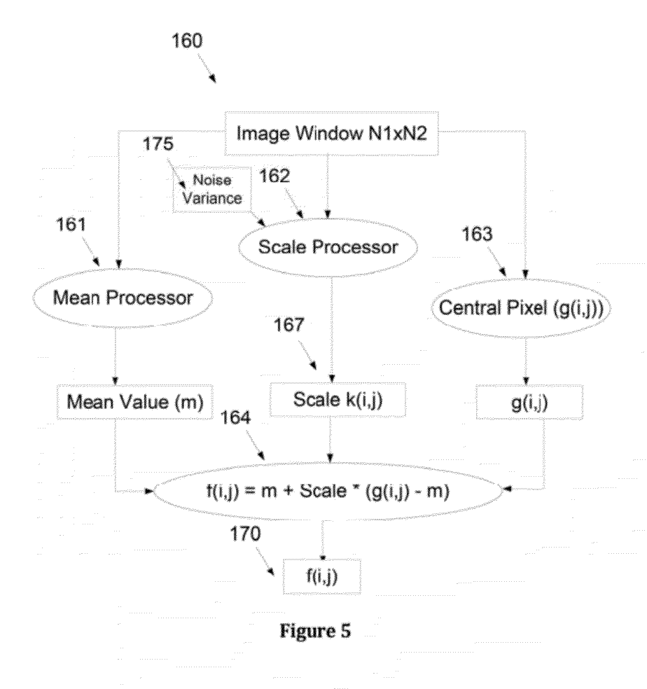

FIGS. 4, 5 and 6 deal with the de-speckle filtering; whose output is DDVS (Down sampled Despeckle Vascular Scan). Speckle noise was attenuated by using a first-order statistics filter (block 160), which gave the best performance in the specific case of carotid imaging. This filter is defined by the following equation (block 164):

J_{x,y}=Ī+k_{x,y}(I_{x,y}−Ī) (1)

where, I_{x,y} is the intensity of the noisy pixel, Ī is the mean intensity of a N×M pixel neighborhood and k_{x,y} is a local statistic measure. The noise-free pixel is indicated by J_{x,y}, k_{x,y} is mathematically defined

$${k}_{x , y} = \frac{{σ}_{l}^{2}}{{\overline{I}}^{2} {σ}_{l}^{2} + {σ}_{n}^{2}} ,$$

where σ₁² represents the variance of the pixels in the neighborhood, and σₙ the variance of the noise in the cropped image (block 175 and 179). An optimal neighborhood size can be 7×7. Note that the despeckle filter is useful in removing the spurious peaks if any during the adventitia identification in subsequent steps.

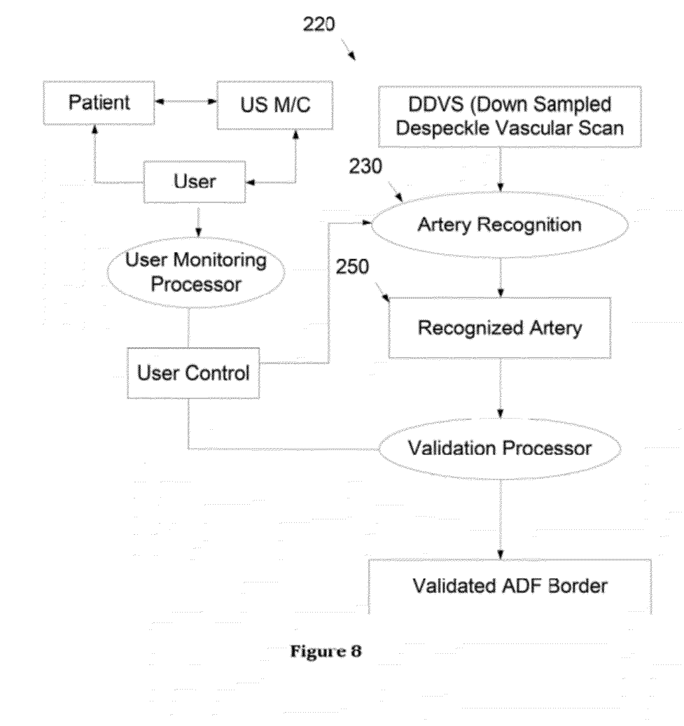

FIG. 8 shows the last and the final stage is the recognition, validation and calibration system shown in the process called “Completely Automated Recognition and Calibration Processor”. While the two stages are cascaded and shown to be different blocks, but it is transparent to the user. This means the software is cascading information from one block to another block without user interaction. The user still has full control and user monitoring processor is fully active and the user can interrupt the system any time. It shows two stages: stage I is the Artery recognition process (block 220) and calibration processor (block 400).

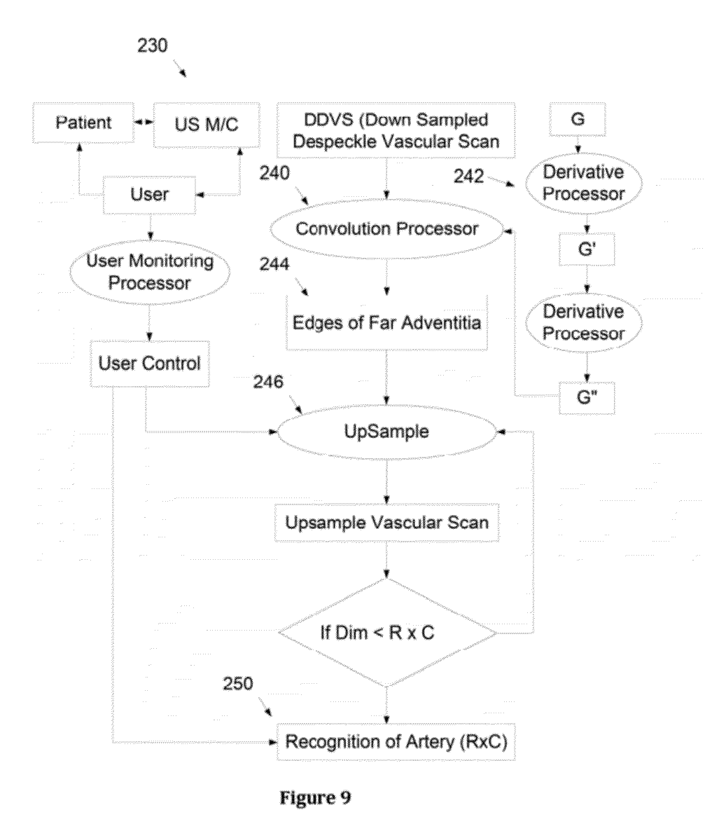

FIG. 9 shows the Artery Recognition Processor (block 220), which is the novelty of the patent as it does the automated recognition. It has two stages: (a) convolution and heuristic processor and (b) up-sample processor.

The convolution processor (block 240) is used for convolution of the first order derivative G (block 242) with the despeckled image. Those skilled in the art can use higher order derivatives as well. The scale parameter of the Gaussian derivative kernel was taken equal to 8 pixels, i.e. to the expected dimension of the IMT value. In fact, an average IMT value of say 1 mm corresponds to about 16 pixels in the original image scale and, consequently, to 8 pixels in the coarse or down sampled image. The convolution processor outcome will lead to the clear information for the near and far walls. This information will have two parallel bands corresponding to the far and near vessel walls. These bands will follow the curvature of the vessel walls. If the vessel wall is oriented downwards or upwards or has a bending nature, the bands will follow on both sides of the lumen. These bands have information which corresponds to the maximum intensity saturated to the maximum values of 2 power 8, the highest value. For an 8 bit image, this value will be 255.

The convolution process then allows the heuristics to estimate the Adventitia borders of the far wall or near wall (block 244). To automatically trace the profile of the far wall, this application uses heuristic search applied to the intensity profile of each column. Starting from the bottom of the image (i.e. from the pixel with the higher row index. The image convention uses (0,0) as top left hand corner of the image), we search for the first white region constituting of at least 6 pixels of width. The deepest point of this region (i.e. the pixel with the higher row index) marked the position of the far adventitia (ADF) layer on that column. The sequence the points resulting from the heuristic search for all the image columns constituted the overall automated far adventitia tracing ADF. The ADF is up sampled back to the original scale (block 246).

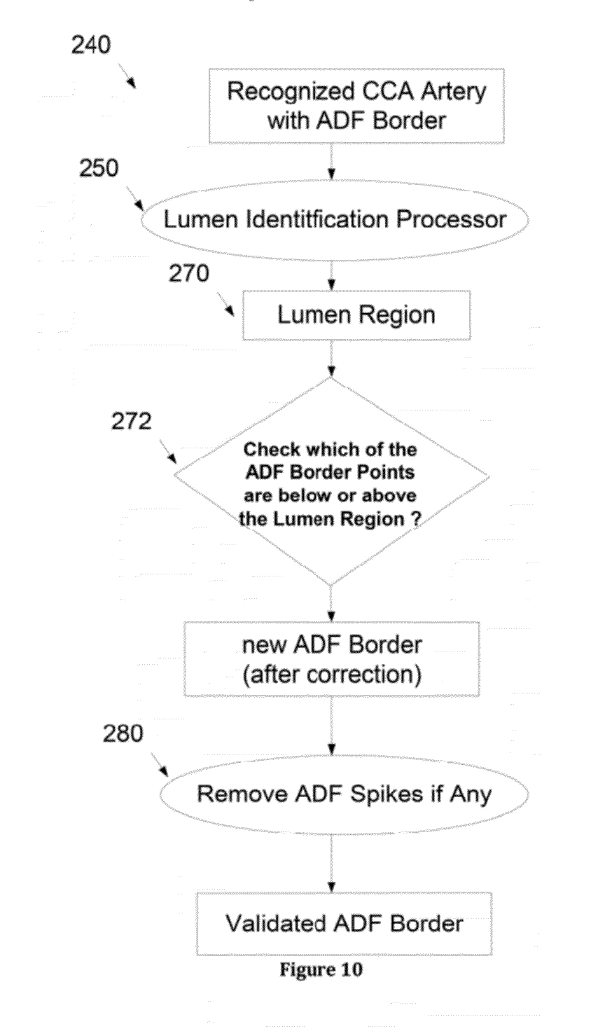

FIG. 10 shows the Artery Validation Processor. Pilot studies showed that the traced ADF profile could be characterized by spikes and false points identification. This could be due to several reasons such as variations in intensities, gaps in the media walls, presence of jugular vein, shadow effects or combination of these. We have therefore introduced a validation protocol, which provides a check on the ADF profile ensuring that the location of CA is at correct place and the far adventitia segmentation edge is not very bumpy. This validation step refines the ADF profile and is done in two steps: (a) refinement using anatomic lumen and (b) spike removal.

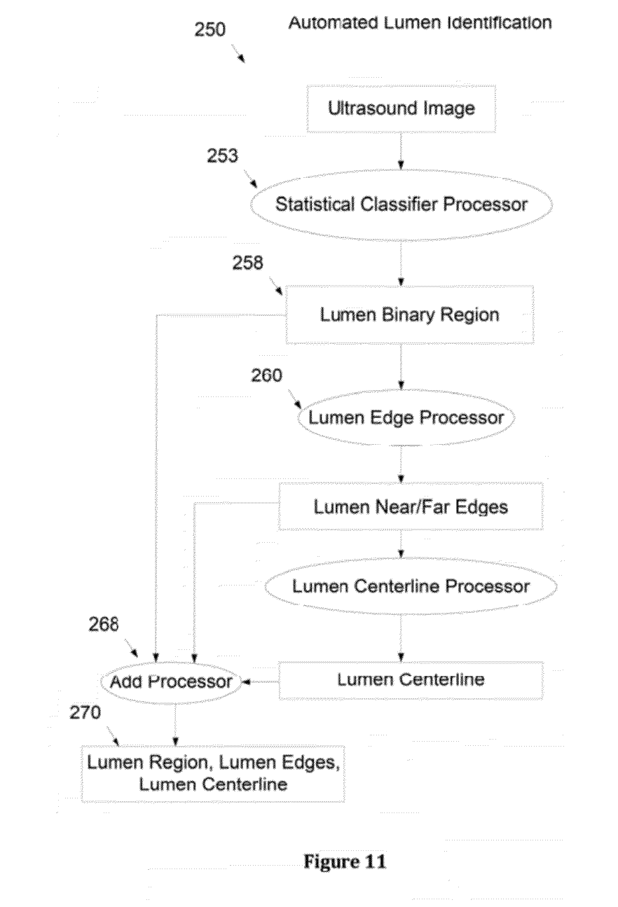

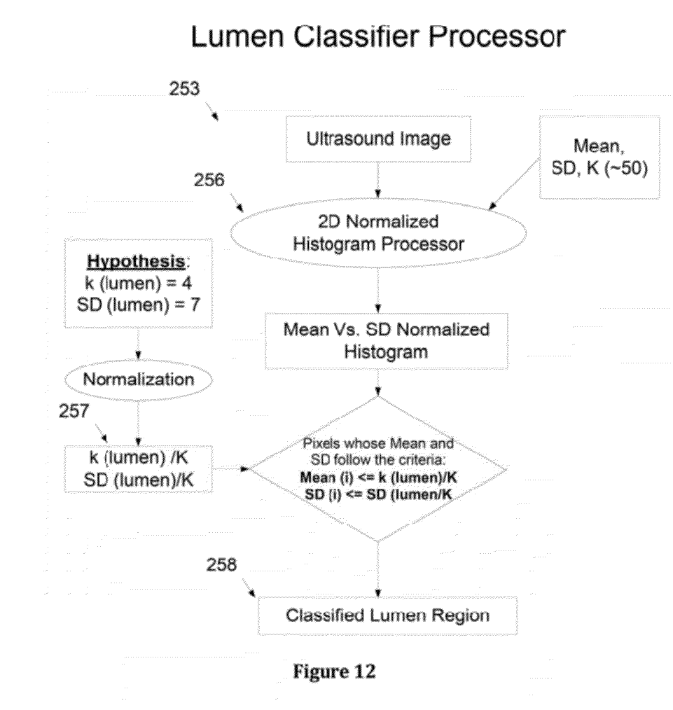

FIG. 11 & FIG. 12 shows the Check system (block 240), which has Lumen Identification Processor (block 250). This check (block 272) has been introduced to avoid error conditions of ADF profile protruding into the lumen vessel or beyond. Thus, the refinement step first requires the identification of the lumen region automatically. We have modeled the lumen segmentation region as a classification process (block 253) with two classes. Carotid characteristics can be thought of as a mixture model with varying intensity distributions. This is because (a) the pixels belonging to the vessel lumen are characterized by low mean intensity and low standard deviation; (h) pixels belonging to the adventitia layer of the carotid wall is characterized by high mean intensity and low standard deviation; and (c) all remaining pixels should have high mean intensity and high standard deviation. As a result of this distribution, we derived a bi-dimensional histogram (2DH) of the carotid image (block 256). For each pixel, we considered a 10×10 neighborhood of which we calculated the mean value and the standard deviation. The mean values and the standard deviations were normalized between 0 and 1 and were grouped into 50 classes each having an interval of 0.02 (block 257). The number of classes equal to 50 and the corresponding class width of 0.02 had been optimized in a previous study. The 2DH was then a joint representation of the mean value and standard deviation of each pixel neighborhood.

In previous studies, we showed that pixels belonging to the lumen of the artery are usually classified into the first few classes of this 2DH: expert sonographer manually traced the boundaries of the CCA lumen and observed the distribution of the lumen pixels on the 2DH. Overall results revealed that pixels of the lumen have a mean values classified in the first 4 classes and a standard deviation in the first 7 classes. We therefore consider a pixel as possibly belonging to the artery lumen if its neighborhood intensity is lower than 0.08 and if its neighborhood standard deviation is lower than 0.14. This shows how the local statistic is effective in detecting image pixels that can be considered as belonging to the CCA lumen. This segmented lumen region act as a check point for the ADF profile estimated before. We therefore utilize the lumen region as follows:

The ADF points along the CA are considered one by one. For each ADF point:
(a) Region of Interest Estimation (Rah): We consider the sequence of the 30 pixels above it (i.e., the 30 pixels located above the AD_{F} point, towards the top of the image, and, therefore, with lower row indexes).
(b) Failure of AD_{F} profile point: We test if the ROI_{L} drawn around the AD_{F} profile points cross the lumen region and have penetrated into the lumen region by at least 15 pixels or more (let's indicate this threshold value by T_{L}). If this does not happens, then the AD_{F} profile point is considered to have failed the lumen test. Pilot experiments we conducted revealed that suitable values for T_{L} are comprised between 12 and 20 pixels.

(c) Tagging of Profile Points: These failed AD_{F} profile points must not belong to the AD_{F} boundary. These AD_{F} points which fail the lumen test are tagged as 0, while rest of the points are tagged as 1. AD_{F} All the AD_{F} points that tagged as 0 are deleted from the AD_{F} list.
(d) The procedure is repeated for each AD_{F} point along the CA artery.

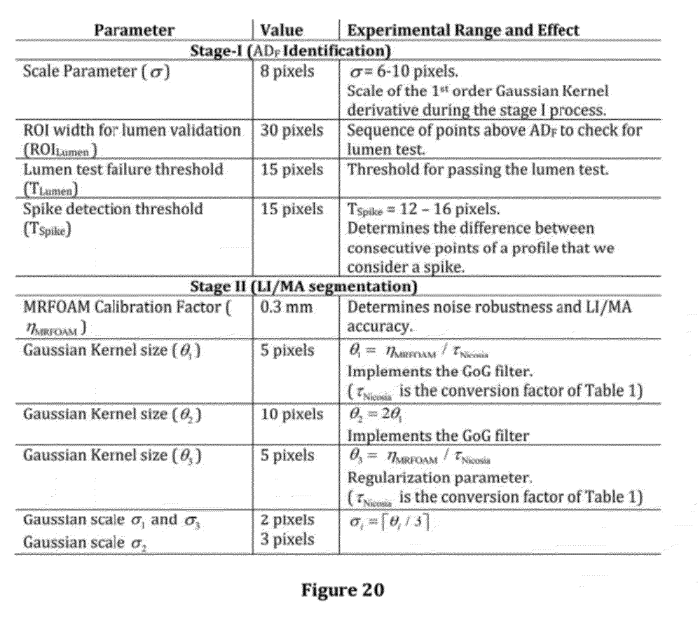

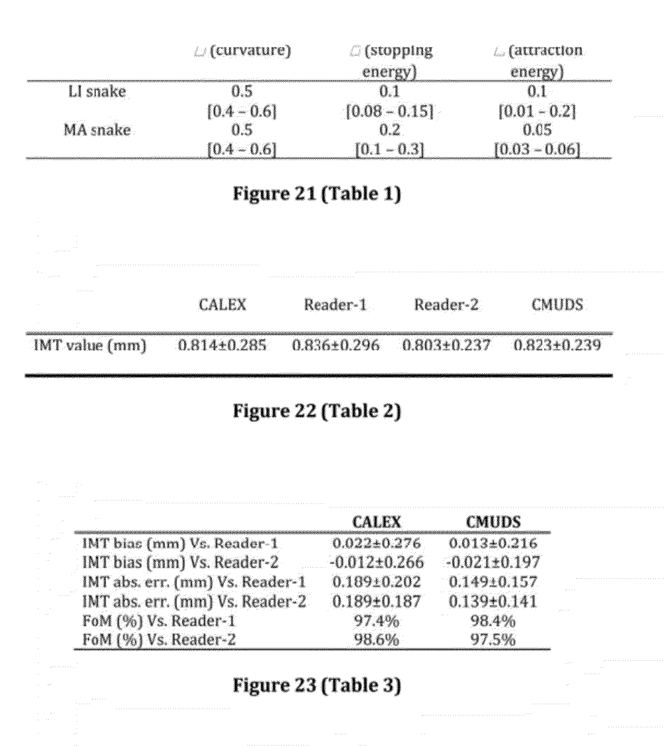

Table 1 in FIG. 20 summarizes all the thresholds and parameters we used in AtheroEdge™. Table 2 in FIG. 21 summarizes the parameters used for Constrained Snakes for LIMA border segmentation. Table 3 in FIG. 22 shows the comparison of the 1MT values between the CMUDS vs. Ground Truth values (Readers). Table 4 in FIG. 23 shows the performance errors of CMUDS vs. CALEX (J Ultras Med 2010a, 29:399-418).

### Spike Detection and Removal.

We implemented an intelligent strategy for spike detection and removal. Basically, we compute the first order derivative of the ADF profile and check for values higher than TS=15 pixels. This value was chosen empirically by considering the image resolution. When working with images having approximate resolution of about 0.06 mm/pixel, an IMT value of 1 mm would be about 12-16 pixels. Therefore, a jump in the ADP profile of the same order of magnitude of the IMT value is clearly a spike and error condition. If the spike is at the very beginning of the image (first 10 columns) or at the end (last 10 columns), then the spiky point is simply deleted. Otherwise, all spikes are considered and either substituted by a neighborhood moving average or removed.

The last stage of the Artery Recognition Processor is the up-sampling processor which allows the adventitia tracing ADF to be up-sampled back to the original scale of cropped image. The ADF profile was then up-sampled to the original scale and superimposed over the original cropped image for both visualization and determination of the region of interest for segmentation (or calibration) phase. At this stage, the CA far wall is automatically located in the image frame and automated segmentation is made possible.

This Artery Recognition Processor (stage-I) is the most innovative aspect of our methodology. It consists of a superior architecture based on fine to coarse sampling for vessel wall scale reduction, speckle noise removal, and higher-order Gaussian convolution, and automated validation embedded recognition of Adventitia. The ability of segmentation or calibration phase (stage-II) to be guided by the automated CA wall recognition is in itself a novel contribution. The first-order Gaussian kernel convolution allowed for an optimal detection of the CA walls. This kernel has unitary energy. When such kernel is located in proximity of a neat gray level change, it enhances the transition. Consequently, the most echoic image interfaces are enhanced to white in the tittered image. For this reason, the Artery Recognition Processor allows for detecting the adventitia layer. This Artery Recognition Processor several advantages to it:
(1) Robustness and Accurate Wall Capture: it is very robust because the higher order derivative kernels are very good in capturing the vessel walls (see, A Review on MR Vascular Image Processing Algorithms: Acquisition and Pre-filtering: Part I, Suri et al., IEEE TRANSACTIONS ON INFORMATION TECHNOLOGY IN BIOMEDICINE, VOL. 6, NO. 4, pp. 324-337, DECEMBER 2002; and A Review on MR Vascular Image Processing: Skeleton Versus Nonskeleton Approaches: Part II, Suri et al., IEEE TRANSACTIONS ON INFORMATION TECHNOLOGY IN BIOMEDICINE, VOL. 6, NO. 4, DECEMBER 2002).
(2) Faster than the conventional processing: Since the recognition is strategized at coarse level down sampled twice from its original size of the image, it is therefore processing ¼^{th} the number of pixels for automated recognition of the media layer. This improves the speed of the system.
(3) Independent of Orientation of vascular scan: Another major advantage to the system is that these Gaussian kernels are independent of the orientation of the blood vessels in the image. Since the ultrasound vascular scans do not always have the vessel orientation horizontal with respect bottom edge of the image, manual methods can pose a further challenge towards the region of interest estimation.
(4) Guiding Method for the calibration System: Since the recognition is followed by the calibration process, the calibration system becomes very robust since the calibration processing is done in the region of interest determined by the automated recognition system. Thus the calibration system adds the value determined by the automated recognition system for vascular ultrasound such as IMT measurement for carotid, femoral, aortic and brachial. Such a combination where the calibration system is guided by the automated recognition system helps in mass processing of huge database processing.
(5) Running the Mass IMT system for Clinical Analysis: Since the recognition is automated followed by the calibration system, the largest value such a system would deliver will be in its real time use for analysis of IMT measurement on a large databases. Running clinical databases on still images would be even more beneficial because such a system would be completely automated in terms of recognition and IMT measurement.
(6) Applications: Since the ultrasound probes use almost the same frequency of operation for scanning the vascular arteries such as carotid, femoral, brachial and aortic, it is thus possible to use such a system for these blood vessels.
   Calibration Processor (stage II)

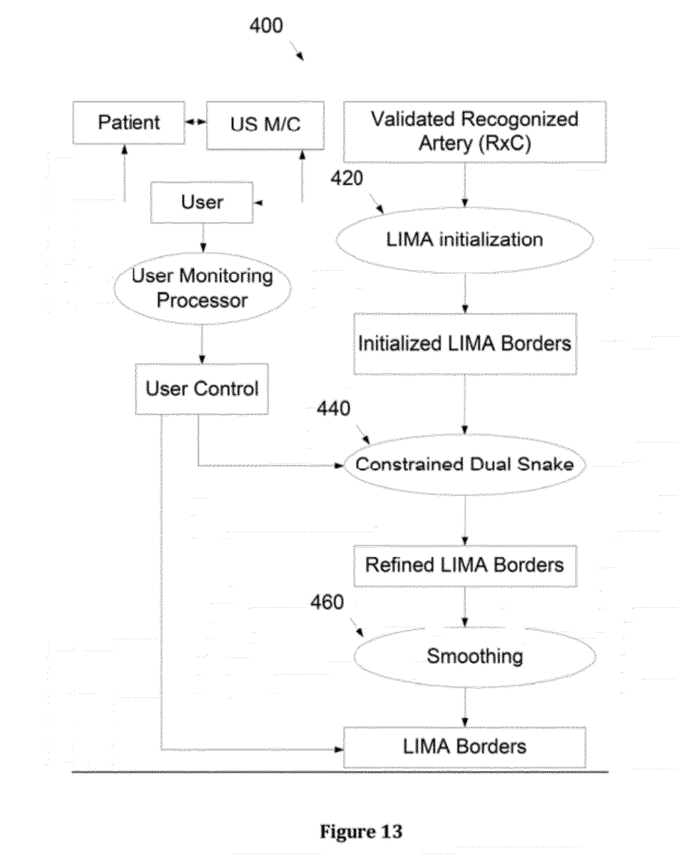

FIG. 13 shows the Calibration Processor (block 400). The output of the multi-resolution strategy provides the automated recognition of the distal wall in the carotid B-mode ultrasound images. Stage-II is focused narrowly in the region of interest, where the objective is to estimate the LIMA borders accurately. The system consists of (a) LIMA Initialization Processor (block 420); (b) Constrained Dual Snake Processor (block 440) and (c) Smoothing Processor (block 460). The goal of stage II i.e., segmentation process is the extraction of the LI and MA borders which lie in between the computed AD_{F} border and the lumen region.

Initialization Processor (stage II)

The AD_{E}: profile was then used to initialize the snakes. The LI snake was initialized by shifting the AD_{F} profile 3 mm upwards. Let's call this Value as Δ_{LI}. Since the maximum IMT value we had in our database was about 1.7 mm, a Δ_{LI} equal to 3 mm ensured that the LI snake was placed into the carotid lumen. The MA snake was initialized by shifting upwards the AD_{F} profile of a value Δ_{MA} equal to 0.1 mm. These values were the optimal choice to place the initial MA snake close to the interface without placing it in-between the LI and the MA boundaries.

The automated initialization procedure was based on the AD_{F} profile for both LI and MA snakes. The LI snake was initialized by the AD_{F} profile that was shifted of Δ_{MA} upwards. Similarly, the initial contour of the MA snake was equivalent to the AD_{F} profile shifted of Δ_{MA} upwards. Therefore, the two snakes initially had the same shape. Then, each snake evolved according to their respective energies. We observed that two possible errors could be presented when:

1. The LI and the MA snake collapse on each other;

2. The LI and the MA snake bleed more than 3 mm.

Calibration Processor (stage II)

As discussed in the above, the IMT is computer-based method for distance estimation between the LI and the MA profiles. Therefore, the image processing strategy must be able to trace the profile of the interface between the artery lumen and the intima layer (LI) and that between the media and the adventitia layers (MA).

Snakes, when used as deformable techniques, are usually initialized close to LI/MA interfaces. They must then evolve until their final shape is exactly the profile as interpreted by the reader (say ultrasound sonographer or vascular surgeon). In our image processing technique, this profile generation is totally automated. This means that the initialization, evolution, and final convergence are all carried Out without any intervention by the user.

A 2-D parametric deformable model (snake) is a mathematical description of a contour evolving on the image. There are two kinds of energies which are responsible for the evolution: (i) Internal energies—which impose the shape smoothness of the contour; such energies directly derive from the mathematical model of the snake itself; (ii) External energies—used to attract the snake in correspondence to the image discontinuities.

Let ν(s) represent the snake, a 2D contour, where the parameter s is the curvilinear coordinate on the image. The curvilinear coordinate s is space-normalized in the range [0,1]. The typical energy functional of a snake ν(s) can be expressed as:

$$E ( v ( s ) ) = {\vert {v}^{′} ( s ) \vert}^{2} + • e ( v ( s ) ) + • \vert v ( s ) • e ( v ( s ) ) \vert ds \qquad ( 1 )$$

In eq. (1), the internal energy Eᵢ consists of the first term:

$${E}_{i} ( v ( s ) ) = {\vert {v}^{′} ( s ) \vert}^{2} ds \qquad ( 2 )$$

Whereas the external energy Eₑ is:

$${E}_{e} ( v ( s ) ) = e ( v ( s ) ) + • \vert v ( s ) • e ( v ( s ) ) \vert ds \qquad ( 3 )$$

The internal energy is used to constrain the shape of the contour and its properties. Specifically, the term □|ν′(s)|² prevents the snake from presenting an excessive curvature. The parameter α is used to control the curvature, whereas ν′(s) is the first-order derivative of the snake curve ν(s). The square modulus is needed in order to make this term energy.

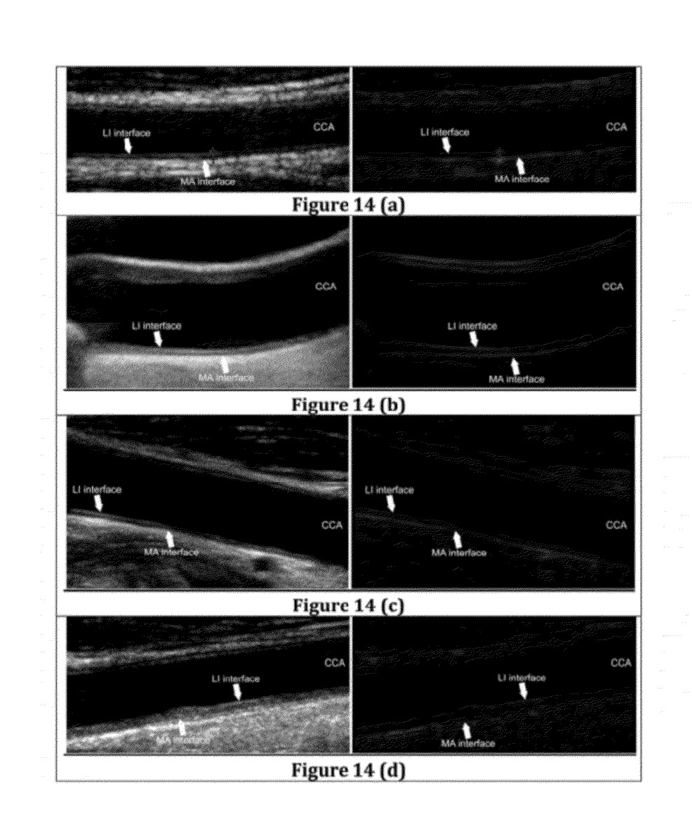

The external energy is used to attract the snake towards the image discontinuities. The first term of the external energy □e(ν(s)) is used in capturing the LI/MA interface of the far wall. In fact, the functional e(x,y) is an edge operator called FOAM (First Order Absolute Moment), which was proposed by Demi et al. [29] and adapted by Faita et al. for detection of the LI/MA edges [11]. FIG. 14 (a), FIG. 14 (b), FIG. 14 (c) and FIG. 14 (d) shows four example of running the FOAM operator on the ROI image (obtained from stage I). This first term of the external energy acts as a stopping function: when the snake points are in correspondence to the image edges, this energy has a high weight and therefore the points tend to be blocked there. The parameter □ controls the strength of this stopping force.

It must be noted that this energy term □e(ν(s)) is null when the snake points are placed far from the edges, where the FOAM operator is equal to zero. Therefore, we added a second term of external energy to our deformable model. The second term of the external energy □|ν(s)□e(ν(s))| is an attraction term, which attracts the snake points when they are far from the edges. This term computes the distance (i.e., the difference) between the edges e(x,y) of the image and the snake position. Therefore, even if the snake is placed in a region where the FOAM is null, it is attracted by the closest edge of the FOAM itself, with an attraction force that is proportional to the distance of the snake from the edge. This term is always positive when the snake is far from the edges, whereas it becomes zero valued when the snake is found in correspondence to the edges. The joint action of the two terms of external energy attracts the snake to the edges and locks the snake once it has reached its stable position.

In our segmentation problem, the edges correspond to the intensity discontinuities of the LI and MA interfaces. Therefore, we used two snakes, one to trace the LI profile and one to trace the MA. Therefore, we had a total of six parameters: three for the LI snake (□_{L1}, □_{L1}, and □_{L1}) and three for the MA snake (□_{MA}, □_{MA}, and □_{MA}). Table 2 (FIG. 21) summarizes the parameter values. We chose the parameter values after pilot tests on a reduced subset of 20 images randomly selected from our database (results not reported here). We had to impose different parameters for the LI and the MA snake because, like Faita et al. [11] pointed out, the LI and MA edges have different intensities in the FOAM edge map. The CMUDS system was very robust and we tested the snake sensitivity to the parameters change. In Table 1 (FIG. 21) we reported the variation range in which the snake performance did not change substantially.

To prevent the snakes from falling in either of the two error conditions, we introduced a mutual constraint that forced the snakes to maintain a constrained distance from one other. We measured the distance between each point of the LI snake (called ν_{L1}(s)) and the line segments of the MA snake (called ν_{MA}(s)). We used the Polyline distance (PDM) as distance metric [34]. Let's call this distance as D_{L1-MA}. We also measured the distance between the points of the MA snake and the line segments of the LI snake and called the distance as D_{MA-L1}. Therefore, the two distances were defined as:

D_{L1□MA}=PDM(ν_{L1})(s),ν_{MA}(s))

D_{MA□L1}=PDM(ν_{MA}(s), ν_{L1}(s)) (4)

When we observed that one point of the LI snake ν_{L1}(s) had a distance higher than 3 mm or lower than 0.3 mm (which we considered as a bottom threshold for IMT) from ν_{MA}(s), we shifted that point up or down until its D_{L1-MA} value was within the imposed constraints. The same correction was applied to the points of the MA snake ν_{MA}(s).

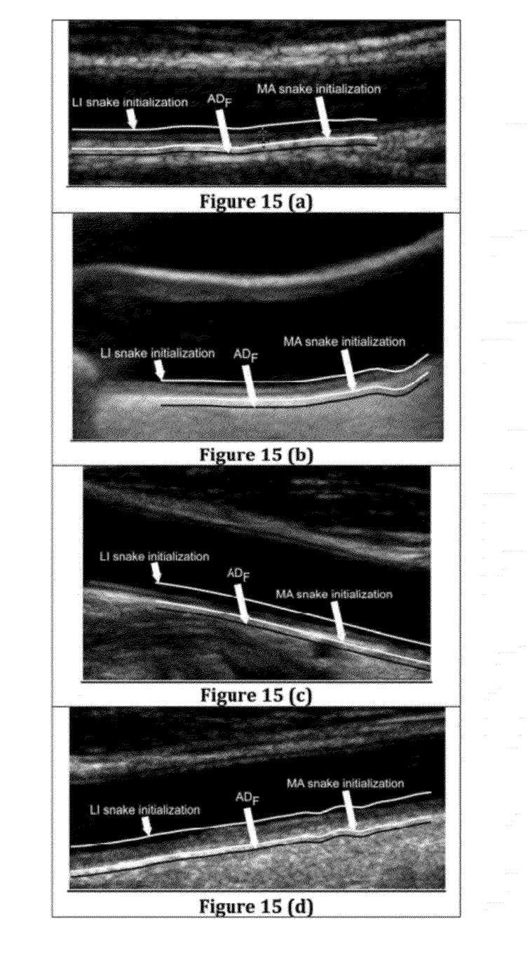

Once automatically initialized, the snakes evolved (without any interaction by the operator) according to the internal and external energies they were subject to. At each iteration step, we computed the coordinates of the snake points. When we observed that both the snakes had stopped evolving, we stopped the iteration and the shape of the snake was considered as the LI/MA computer generated profiles. To avoid being trapped in a loop, we set a maximum limit on the number of iterations equal to 200 or convergence, which ever reached first. FIG. 15 (a) is an example of initialization and FIG. 15 (b) shows the CMUDS segmentation.

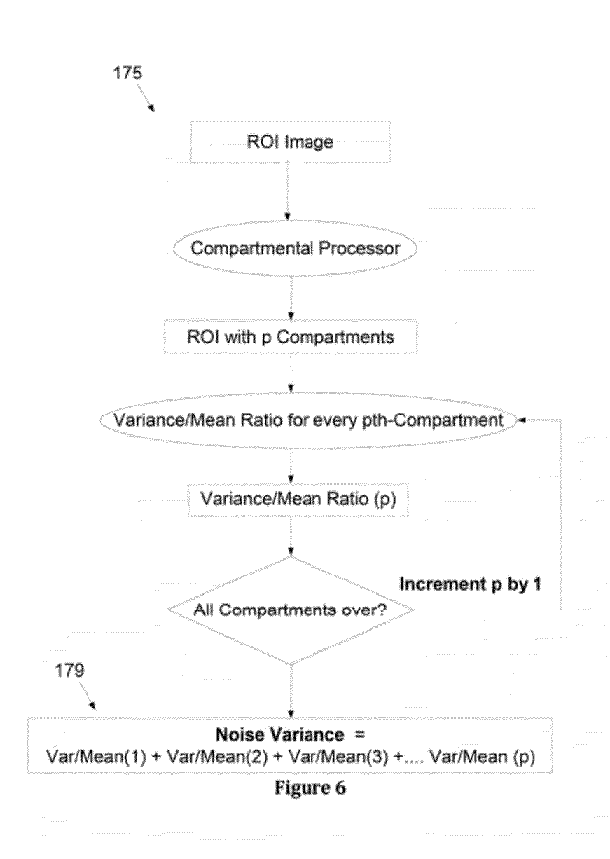

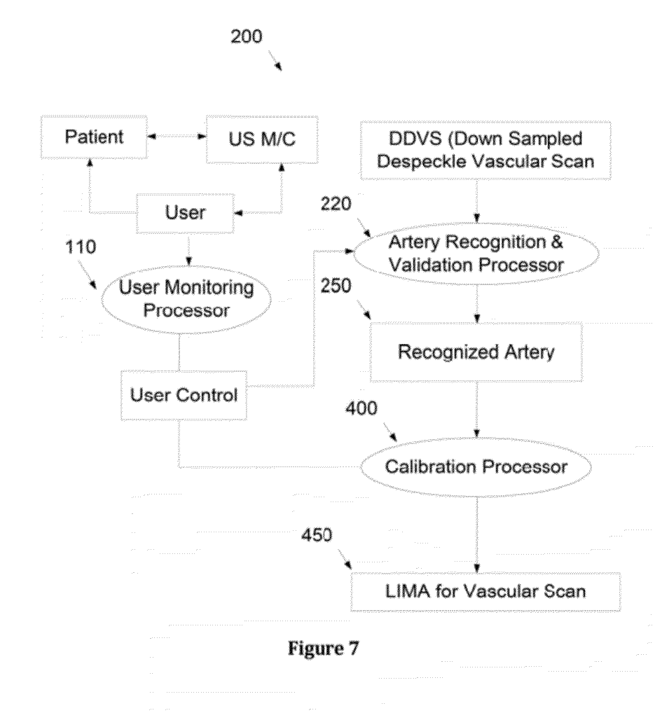

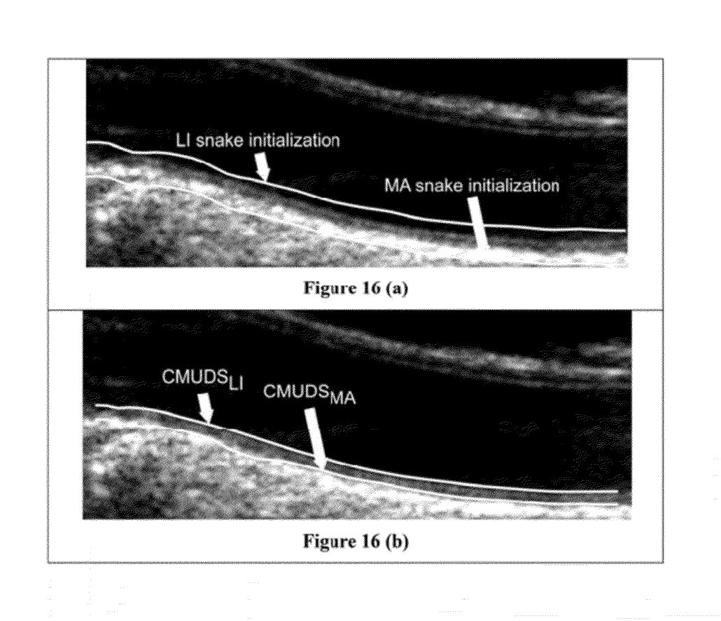

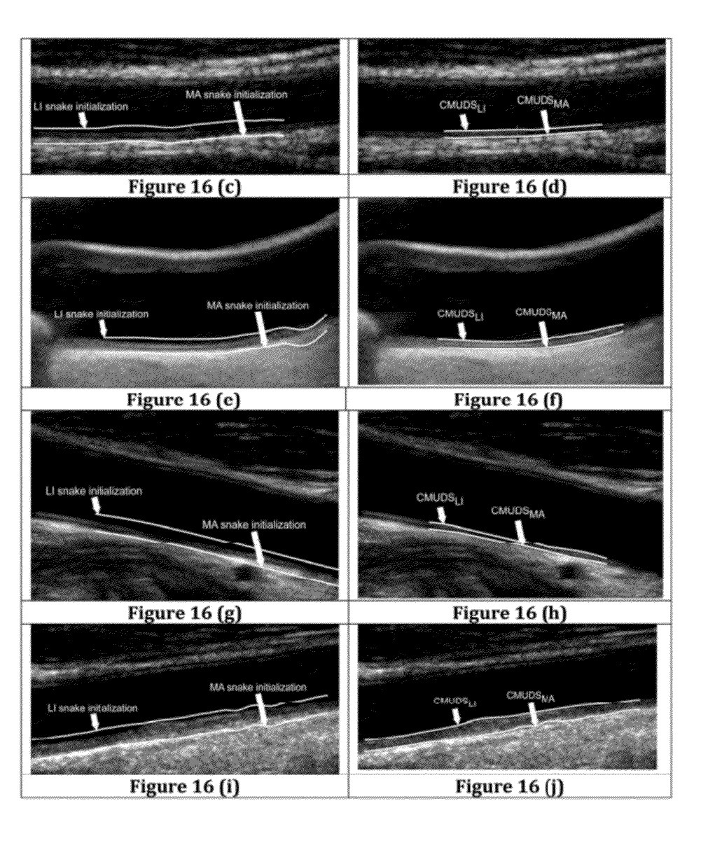

FIG. 16 (a), 16 (c), FIG. 6 (e), FIG. 16 (g), FIG. 16 (i) shows the LIMA initialization borders and FIG. 16 (d), FIG. 16 (f), FIG. 16 (h), FIG. 16 (j) and shows the final segmentation of CMUDS on four different carotid examples.

### CMUDS Evolution and Convergence Examples

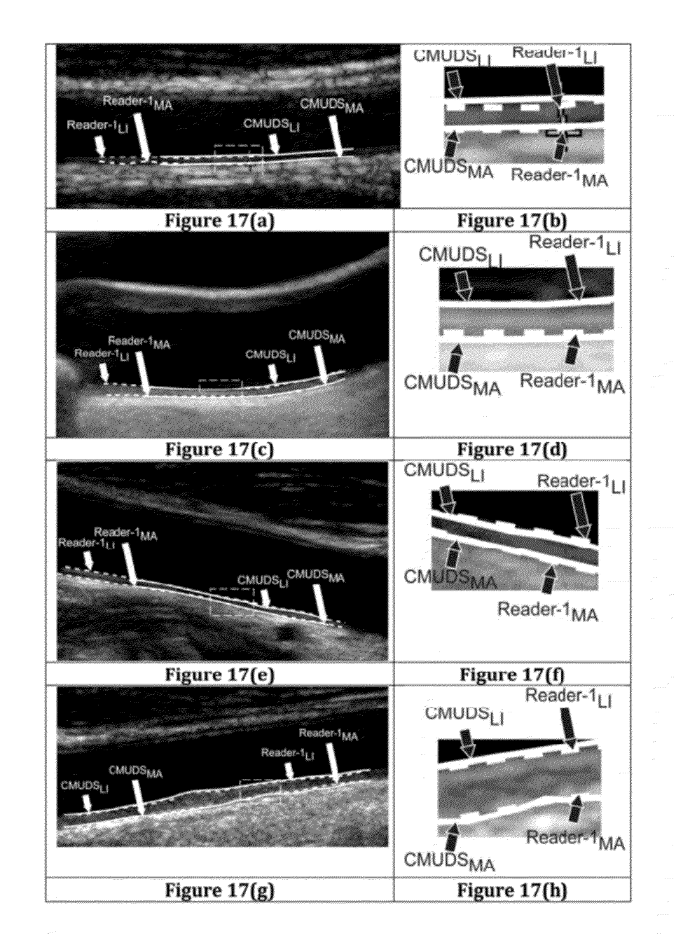

FIG. 17 (a), FIG. 17 (c), FIG. 17 (e) and FIG. 17 (g) shows the LIMA performance with respect the gold standard drawn by the Reader-1. The upper WHITE solid line depicts the LI snake and the lower WRITE solid depicts the MA snake. The zoomed versions are shown in FIG. 17 (b), FIG. 17 (d), FIG. 17 (f) and FIG. 17 (h). The initial average distance between the LI snake and the Reader-1_{L1} profile was equal to 0.44±0.34 mm. At convergence, the distance reduced to 0.025±0.041 mm (p<10⁻²⁰). Ideally, this distance should be zero. The initial distance between the MA snake and the Reader-1_{MA} profile was 0.68±0.35 mm. At convergence, it reduced to 0.023±0.043 mm (p<10⁻⁹⁰). When compared to Reader-2 (or Reader-2), the initial LI distance was 0.46±0.39 mm, which reduced to 0.022±0.038 mm (p<10⁻²²); whereas the MA distance decreased from 0.72±0.38 mm to 0.020±0.037 mm (p<10⁻⁹⁰).

CUMDS's Performance using Figure of Merit:

FIG. 23 (Table 4) summarizes the IMT performance of CMUDS compared to the readers and CALEX (J Ultras Med 2010a, 29:399-418) system, respectively. The table reports the measurement errors compared to the readers' values: the first two row reports the IMT measurement bias and the second-third the IMT absolute error. All the values were compared to Ground Truth or reader tracings. To measure the system performance, we computed the Figure-of-Merit (FoM) for CMUDS and CALEX as:

$${FoM}_{CMUDS} = 100 - \vert \frac{{IMT}_{CMUDS} - {IMT}_{Reader}}{{IMT}_{Reader}} \vert \cdot 100 \\ {FoM}_{CALEX} = 100 - \vert \frac{{IMT}_{CALEX} - {IMT}_{Reader}}{{IMT}_{Reader}} \vert \cdot 100 \qquad ( 5 )$$

where IMT_{CMUDS} is the average IMT value as computed by the CMUDS Dual Snake technique, IMT_{CALEX} is the average IMT computed by CALEX, and IMT_{Reader} is the average IMT when the tracings were done manually.
The FoM of the two systems is reported in the last row of FIG. 23 (Table 3). It is possible to observe that when compared to Reader-1, CMUDS outperformed CALEX and obtained a FoM equal to 98.4% against 97.4% of CALEX. However, when compared to Reader-2, CALEX was more accurate and obtained a FoM of 98.6% against 97.5% of CMUDS. However, we found that CMUDS average IMT was not statistically different from either Reader-1 (p>0.15) or Reader-2 (p>0.13), whereas the CALEX IMT values were significantly different from those of Reader-1 (p<0.01) and Reader-2 (p<0.03). Also, the absolute NT measurement errors (third row of Table 3) were lower for CMUDS than for CALEX, both in the comparison with Reader-1 and Reader-2 (p<0.009 for both tests). This happened because the computer tracings by CMUDS were more globally closer to the readers' tracings with respect to the tracings by CALEX. By summarizing, CMUDS was more accurate than CALEX. We also observed that the standard deviation of the IMT error was statistically lower for CMUDS than for CALEX, both in the comparison with Reader-1 (p<10⁻⁸) and with Reader-2 (p<10⁻¹¹). Hence, CMUDS was also more reproducible than CALEX.
CUMDS's Performance using Bland-Altmann Plots:

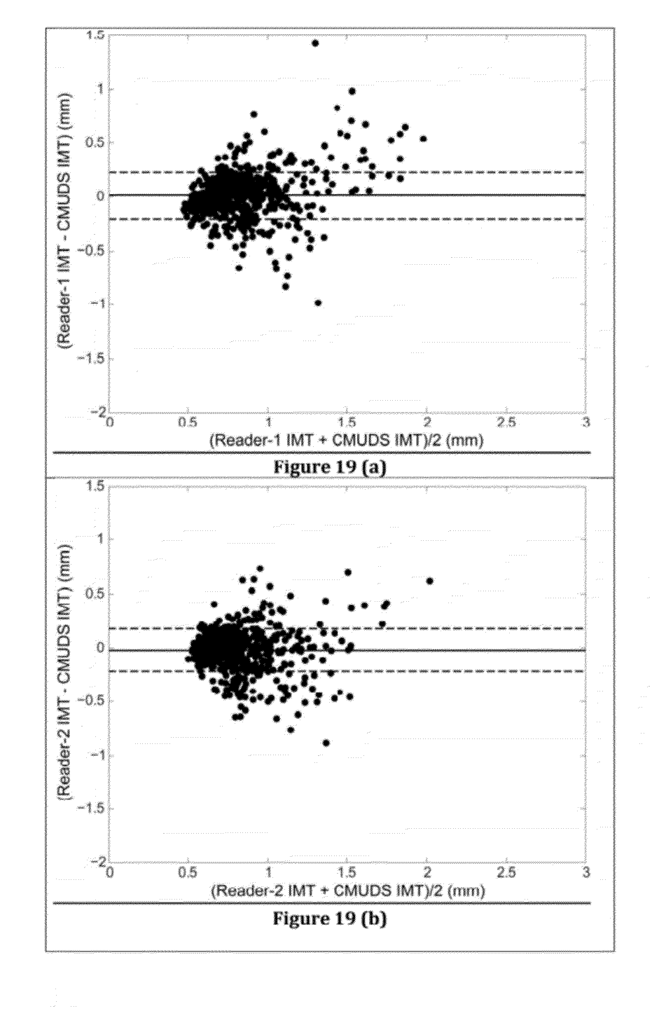

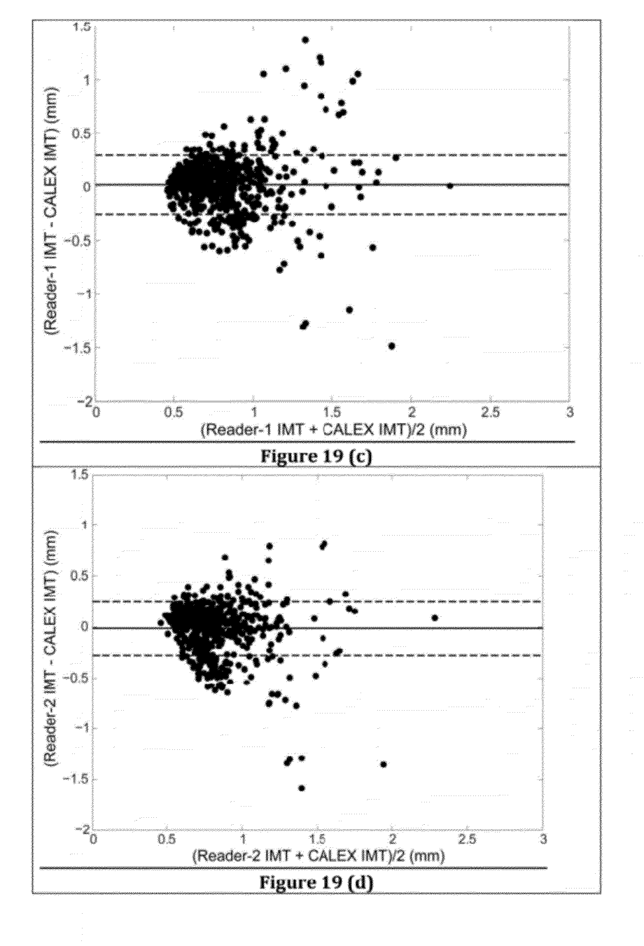

FIG. 19 (a), FIG. 19 (b), FIG. 19 (c), and FIG. 19 (d) shows the Bland-Altmann plots for CMUDS and CALEX IMT measurements. It can be noticed that there is the absence of any bias and the measurements are all very close to the manual values. FIG. 19 (a) and FIG. 19 (h) shows the Bland-Altmann plots for CMUDS and FIG. 19 (c) and FIG. 19 (d) for CMUDS. The graphs on the left are relative to the comparison against the Reader-1, the graphs on the right are relative to the comparison against the Reader-2.

### Data Interpretation

We have developed a fully automated computer-based system for the tracing of the LI and MA profiles of the carotid distal wall artery from longitudinal ultrasound 13-Mode images. This system is unique and novel in the medical imaging scenario for several reasons:
a) It is a Dual Snake system, which means that two deformable parametric models (one for the LI and one for the MA) are initialized simultaneously and converge jointly step-by-step. The LI and MA profiles are constrained during the evolution process.
b) It is automatically initialized starting from the far adventitia profile;
c) It has an external energy (attraction force) that depends on the FOAM operator rather than on the conventional image gradients.
d) Easily adaptable to clinical settings.
e) Highly accurate and reproducible system.
   No other methodologies that we are aware of use a Dual Snake system for LI/MA interface computation and IMT measurement. Usually, snake-based methods use the snake sequentially in time, by first running it for LI interface computation, followed by MA computation. The evolution of the snakes is, therefore, independent. Our system can be considered as a Dual Snake, because our LI/MA snakes evolve simultaneously and are constrained by each other. This constraint prevents the snakes from bleeding or collapsing.

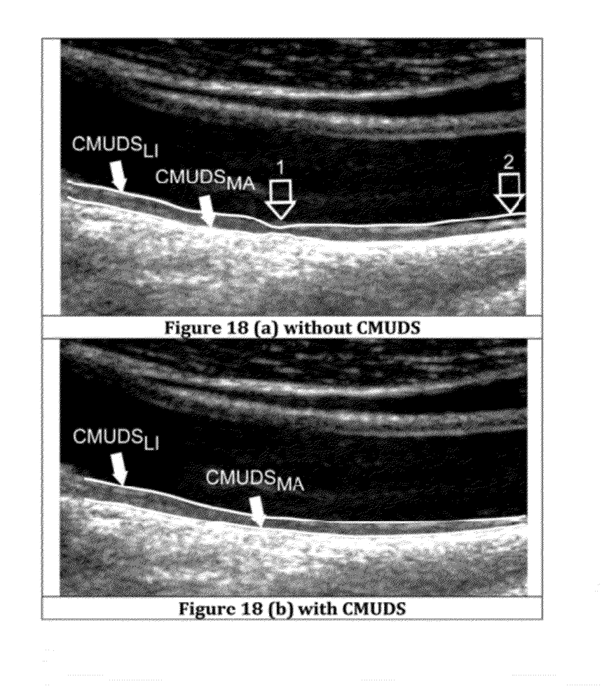

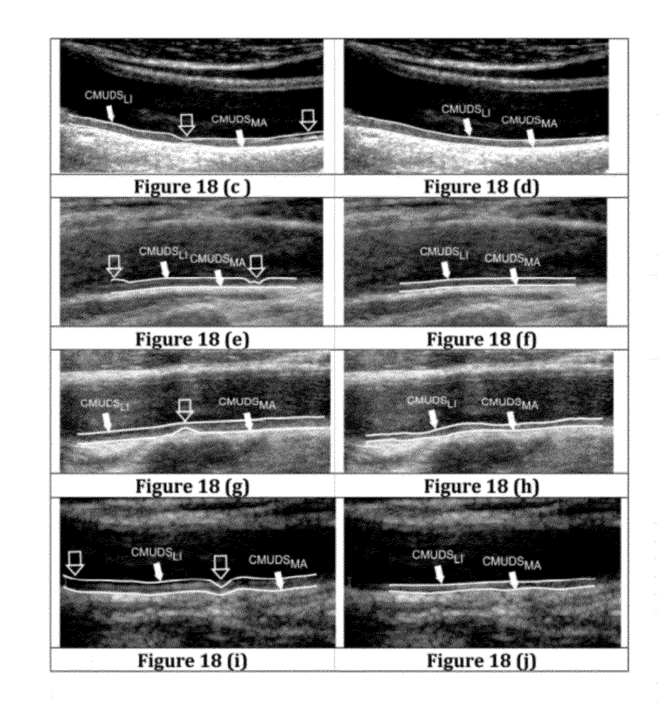

FIG. 18 (a) and FIG. 18 (b) shows the effect of the mutual constraint between the LI and the MA snakes. FIG. 18 (a) shows the CMUDS final segmentation of a slightly curved carotid. We ran CMUDS system again on the same image, but after having removed the mutual constraint (i.e., the LI and MA snakes were allowed to converge independently one on the other). The obtained segmentation without mutual constraint is shown by FIG. 18 (b). The white arrows indicate image regions in which the tracings by the snakes are incorrect. The arrow marked as “1” shows that the LI and MA snakes nearly collapsed. The arrow marked as “2” indicates the LI snake bleeding from the actual interface. Hence, our mutually constrained dual snake system can be thought as a major technical improvement to ensure stability and accuracy to the CMUDS system.

The best performing snake-based algorithms that were published in literature were by Cheng et al. [24], Loizou et al. [26], and Delsanto et al. [25]. All these techniques segmented the LI/MA profiles independently thereby more susceptible to noise. Cheng et al. proposed a zip-lock snake. Such snake originates from two points that must be placed very close to the interface one wants to segment [24]. Therefore, manual interaction was required in order to start the snake convergence. Loizou et al. adopted a semi-automated initialization in which the user had to draw a rectangular box in correspondence the carotid distal wall [26]. The lumen and the adventitia had to be contained in this box, so that the snake initialization could be performed by using vertical gradients. Delsanto et al. proposed an automated method for the snake initialization. Such method was based on a user-independent procedure for the carotid lumen detection that was based on signal analysis [25,40,41]. All the pixels that were considered as possibly belonging to the carotid lumen constituted a mask, which was then used to locate the distal artery wall. The LI/MA snakes were initialized by using the vertical gradients [40].

Therefore, only the snake algorithm by Delsanto et al. was completely user-independent. However, compared to that algorithm our CMUDS approach offers three significant advantages. First, it uses FOAM as the external energy. The snake by Delsanto et al. used the image gradients for convergence. However, the traditional image gradients are very sensitive to noise. Hence, when the image signal-to-noise ratio is low, the snake interfaces either bleeds or get trapped in the local minima. This is the typical condition of an artery with a noisy lumen, caused by blood rouleaux. The FOAM operator enhances the image edges and is very robust with respect to noise [11,42]. This ensured a good robustness to noise and artifacts.

The second advantage of CMUDS with respect to the algorithm proposed by Delsanto et al. is given by the presence of the mutual constraint. This prevents the snakes from bleeding, collapsing and from being trapped in-between the LI/MA boundaries (which was a typical error condition of the snake by Delsanto et al., which was described in [43]). Snake trapping or collapsing was not observed in any of the 665 images we processed by our novel CMUDS technique.

The third advantage is the computational time. The MATLAB implementation of Delsanto et al.'s snake required about 32 s (tested on a dual 2.5 GHz G5 PowerPc equipped with 4 GB of RAM) [35]. Our new algorithm is much faster, since the snake convergence is carried out simultaneously. Therefore, by using the same MATLAB environment on the same testing machine, the average processing time of CMUDS was 4.1±1.1 s, while, being still fully automated.

The direct performance comparison between this CMUDS algorithm and previously developed techniques is not straightforward. First of all, only Cheng et al. declared the average biases in the LI and MA tracings. They worked with images having a conversion factor of 0.096 mm/pixel and obtained an average LI error of 0.063 mm and MA error of 0.039 mm. However, they used the root mean squared error as a performance metric and the Euclidean distance as a distance metric [24]. Therefore, results are not directly comparable to CMUDS, because the Euclidean distance strongly biases the distances when the artery is not straight or is not horizontal. Delsanto et al. showed a LI segmentation error equal to 0.035±0.032 mm and a MA error of 0.037±0.029 when the Euclidean distance was used and the testing dataset was single-institution [35]. Our new Dual Snake CMUDS system showed LI errors equal to about 0.025±0.041 mm and MA errors equal to 0.023±0.043 mm (in the worst condition), which were comparable to those of previous techniques.

The best performing computer methods we found in literature was the semi-automated technique by Faita et al. [11]. It which was based on FOAM. They obtained an average IMT measurement error equal to about 0.010±0.038 mm. The FOAM-based technique by Faita et al. was user-driven: the reader had to select a region-of-interest around the distal wall, where the IMT measurement was then performed by using FOAM. We ran the semi-automated FOAM as standalone on our 665 images database and we obtained an average IMT value equal to 0.804±0.243 mm, with an IMT measurement bias equal to 0.001±0.223 mm when compared to Reader-1 and of 0.032±0.255 mm when compared to Reader-2. Therefore, on our multi-institutional database, we could not obtain the same performance Faita et al. obtained on their single-institution dataset. Our explanation is that we worked on a multi-institutional database consisting of images with different pixel density and noise level.

Compared to the automated benchmarking technique (CALEX), CMUDS showed better performance. Despite the fact that the FoM of CMUDS was higher than that of CALEX for Reader-1, but lower for Reader-2, the IMT bias was always better compared to CALEX. CMUDS improved accuracy and reproducibility of the IMT measurement and both the manual tracing sets confirmed this observation. CMUDS system is fully automated and showed good performance on a multi-institutional and multi-ethnic database. Therefore, it could be a suitable technique for the automation of the IMT measurement in large epidemiological and multi-center studies. Touboul et al. in 2005 showed the need for standardization in multi-center studies involving the IMT measurement (PARC study [9]). Recently, Polak et al. [4,7] showed the differences among manual measurements and computer assisted measurements when risk factors were associated to the carotid IMT [4,7]. In this scenario, CMUDS could be a clinically suitable processing system.

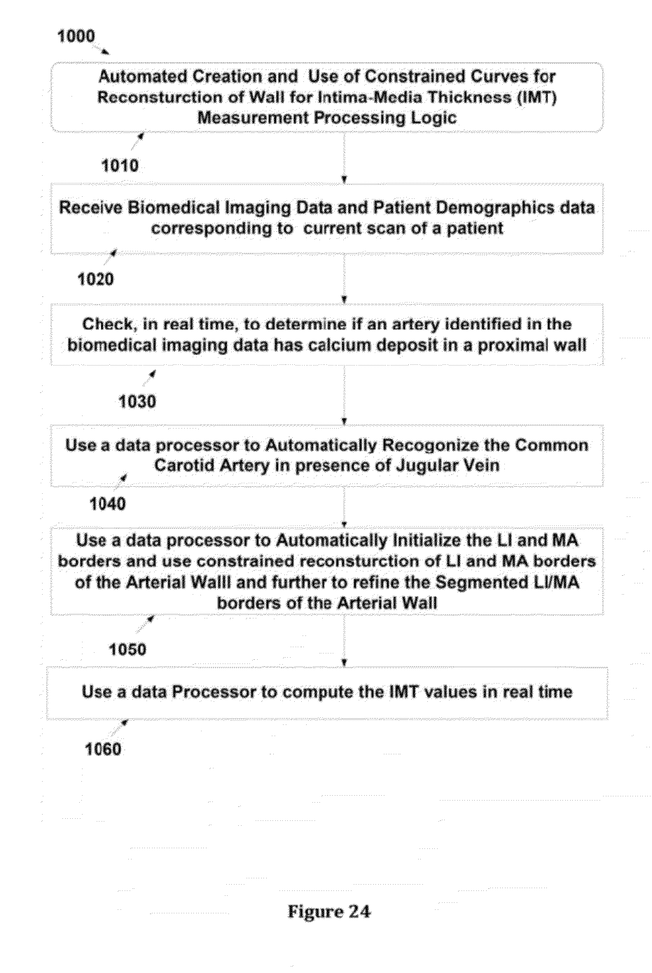

FIG. 24 is a processing flow diagram illustrating an example embodiment of a computer-implemented system and method for intima-media thickness (IMT) measurements using a validation embedded segmentation method as described herein. The method 2600 of an example embodiment includes: receiving biomedical imaging data and patient demographic data corresponding to a current scan of a patient (processing block 2610); checking the biomedical imaging data in real-time to determine if an artery of the patient has a calcium deposit in a proximal wall of the artery (processing block 2612); acquiring arterial data of the patient as a combination of longitudinal B-mode and transverse B-mode data (processing block 2614); using a data processor to automatically recognize the artery by embedding anatomic information (processing block 2616); using the data processor to calibrate a region of interest around the automatically recognized artery (processing block 2618); and determining the intima-media thickness (IMT) of an arterial wall of the automatically recognized artery (processing block 2620).

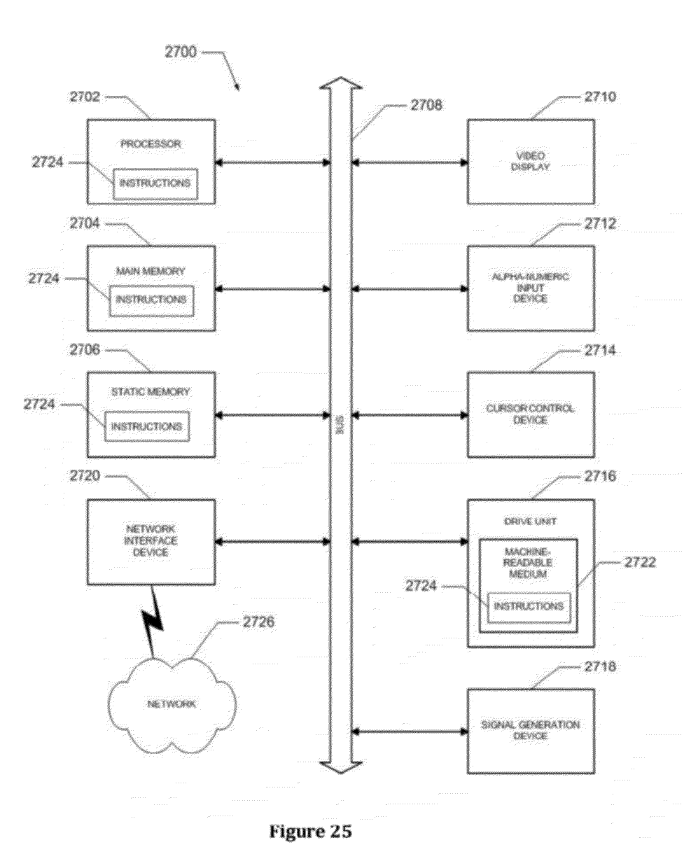

FIG. 25 shows a diagrammatic representation of machine in the example form of a computer system 2700 within which a set of instructions when executed may cause the machine to perform any one or more of the methodologies discussed herein. In alternative embodiments, the machine operates as a standalone device or may be connected (e.g., networked) to other machines. In a networked deployment, the machine may operate in the capacity of a server or a client machine in server-client network environment, or as a peer machine in a peer-to-peer (or distributed) network environment. The machine may be a personal computer (PC), a tablet PC, a set-top box (STB), a Personal Digital Assistant (PDA), a cellular telephone, a web appliance, a network router, switch or bridge, or any machine capable of executing a set of instructions (sequential or otherwise) that specify actions to be taken by that machine. Further, while only a single machine is illustrated, the term “machine” can also be taken to include any collection of machines that individually or jointly execute a set (or multiple sets) of instructions to perform any one or more of the methodologies discussed herein.

The example computer system 2700 includes a processor 2702 (e.g., a central processing unit (CPU), a graphics processing unit (GPU), or both), a main memory 2704 and a static memory 2706, which communicate with each other via a bus 2708. The computer system 2700 may further include a video display unit 2710 (e.g., a liquid crystal display (LCD) or a cathode ray tube (CRT)). The computer system 2700 also includes an input device 2712 (e.g., a keyboard), a cursor control device 2714 (e.g., a mouse), a disk drive unit 2716, a signal generation device 2718 (e.g., a speaker) and a network interface device 2720.

The disk drive unit 2716 includes a machine-readable medium 2722 on which is stored one or more sets of instructions (e.g., software 2724) embodying any one or more of the methodologies or functions described herein. The instructions 2724 may also reside, completely or at least partially, within the main memory 2704, the static memory 2706, and/or within the processor 2702 during execution thereof by the computer system 2700. The main memory 2704 and the processor 2702 also may constitute machine-readable media. The instructions 2724 may further be transmitted or received over a network 2726 via the network interface device 2720. While the machine-readable medium 2722 is shown in an example embodiment to be a single medium, the term “machine-readable medium” should be taken to include a single medium or multiple media (e.g., a centralized or distributed database, and/or associated caches and servers) that store the one or more sets of instructions. The term “machine-readable medium” can also be taken to include any non-transitory medium that is capable of storing, encoding or carrying a set of instructions for execution by the machine and that cause the machine to perform any one or more of the methodologies of the various embodiments, or that is capable of storing, encoding or carrying data structures utilized by or associated with such a set of instructions. The term “machine-readable medium” can accordingly be taken to include, but not be limited to, solid-state memories, optical media, and magnetic media.

The Abstract of the Disclosure is provided to comply with 37 C.F.R. §1.72(b), requiring an abstract that will allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separate embodiment.

What is claimed is:

1. A computer-implemented method comprising:
receiving biomedical imaging data and patient demographic data corresponding to a current scan of a patient's carotid, brachial, femoral and arotic vessels;
real time checking if the artery has atherosclerosis deposit or no atherosclerosis deposit in the proximal or near wall;
acquiring the arterial data as a combination of longitudinal B-mode and transverse B-mode or longitudinal B-mode or longitudinal M-mode or a combination of longitudinal M-mode and transverse M-mode alone;
using a data processor to automatically recognize or locate the artery by embedding anatomic information (stage I);
using the data processor to calibrate the region of interest around the automatically recognized artery using constrained based evolution (stage II); and
determining the IMT of the arterial wall.

2. The method as claimed in claim 1 wherein the biomedical imaging data comprises of combination of two-dimensional (2D) longitudinal B-mode and two-dimensional (2D) transverse B-mode ultrasound images or longitudinal B-mode alone, with and without the presence of the atherosclerosis and applicable to Carotid, Brachial, Femoral and Aorta Vessels.

3. The method as claimed in claim 1 wherein the method can be for automated recognition (stage I) using a multi-resolution approach, where the edges of the MA border are determined in coarse resolution and applicable to Carotid, Brachial, Femoral and Aorta Vessels.

4. The method as claimed in claim 1 wherein the method can be for automated recognition (stage I) using a multi-resolution approach, where the edges of the MA border are determined in coarse resolution and up-sampled back onto the original high resolution image. This is applicable to applicable to Carotid, Brachial, Femoral and Aorta Vessels.

5. The method as claimed in claim 1 wherein the method can be for automated recognition (stage I) using a multi-resolution approach, and the artery location can be validated using anatomic reference information such as lumen and applicable to Carotid, Brachial, Femoral and Aorta Vessels.

6. The method as claimed in claim 1 wherein the method can be for automated recognition (stage I) using a multi-resolution approach, and the artery location can be validated using anatomic information such as lumen. The lumen is automatically located in the image frame using the statistical classifier and applicable to Carotid, Brachial, Femoral and Aorta Vessels.

7. A computer-implemented method comprising:
receiving biomedical imaging data and patient demographic data corresponding to a current scan of a patient's carotid, brachial, femoral and arotic vessels;
real time checking if the artery has atherosclerosis deposit in the proximal or near wall;
acquiring the arterial data as a combination of longitudinal B-mode and transverse B-mode;
using a data processor to automatically recognize the artery;
using the data processor to calibrate the region of interest around the automatically recognized artery; and determining the IMT of the arterial wall; where,
the automated recognition is computed in coarse resolution by convolution of higher order derivative of Gaussian kernels, the scale of the kernel is computed using a priori anatomic information from the database of ultrasound images.

8. The method as claimed in claim 7 wherein the method where the automated recognition using higher order derivative can be with and without atherosclerosis present in the arterial proximal wall.

9. The method as claimed in claim 7 wherein the method where the calibration stage (stage II) is guided by the automated recognition of the artery, where the step one of the calibration stage (stage II) is the automated initialization of far or near LI and MA borders, and applicable to the near and far wall of the Carotid, Brachial, Femoral and Aorta Vessels.

10. The method as claimed in claim 7 wherein the calibration stage (stage II) is guided by the automated recognition of the artery (ADF), where the step one of the calibration stage is the automated initialization of far or near LI and MA borders, where in the initial LI and initial MA is obtained from ADF (far adventitia borders) by shifting the ADF borders. Such a system is applicable to both near and far wall of the Carotid, Brachial, Femoral and Aorta Vessels.

11. The method as claimed in claim 7 wherein the calibration stage is guided by the automated recognition of the artery (ADF), where the step one of the calibration stage is the automated initialization of far or near LI and MA borders, where in the initial LI and initial MA is obtained from ADF (far adventitia borders) is used for starting the evolution of the deformable model. Such a system is applicable to both near and far wall of the Carotid, Brachial, Femoral and Aorta Vessels.

12. The method as claimed in claim 7 wherein the calibration stage is guided by the automated recognition of the artery (ADF), where the step one of the calibration stage is the automated initialization of far or near LI and MA borders, where in the evolution of the LI and MA are constrained. Such a system is applicable to both near and far wall of the Carotid, Brachial, Femoral and Aorta Vessels.

13. The method as claimed in claim 7 wherein the calibration stage is guided by the automated recognition of the artery (ADF), where the step one of the calibration stage is the automated initialization of far or near LI and MA borders, where in, the evolution of the LI and MA borders are constrained using the Polyline Distance Method or Centerline Distance Method or Distance Transform Method. Such a system is applicable to both near and far wall of the Carotid, Brachial, Femoral and Aorta Vessels.

14. The method as claimed in claim 7 wherein the calibration stage is guided by the automated recognition of the artery (ADF), where the step one of the calibration stage is the automated initialization of far or near LI and MA borders, where in, the evolution of the LI and MA borders are simultaneously evolving under the constraints binded by the Polyline Distance Method or Centerline Distance Method or Distance Transform Method. Such a system is applicable to both near and far wall of the Carotid, Brachial, Femoral and Aorta Vessels.

15. The method as claimed in claim 7 where the automated recognition (stage I) can be a feature based method unlike the multi-resolution higher order derivative approach. This automated recognition method guides the stage-II (calibration method) for LI and MA border estimation using constrained snake evolution. Thus, the system for IMT measurement for Carotid, Brachial, Femoral and Aorta Vessels can use a feature-based method or multi-resolution derivative approach in stage I followed by a constrained snake evolution method as stage II.

16. The method claimed in claim 7 where the segmented region between the LI and MA borders (so called Regional IMT wall region) can be used for symptomatic vs. asymptomatic plaque characterization (Atheromatic™).

17. The method as claimed in claim 7, wherein the calibration method can be changed from constrained snake or deformable model to a classification model like mean shift classifier or Bayesian classifier or fuzzy classifier or Heuristic based approach.

18. The method as claimed in claim 1 wherein the method can be used for monitoring the IMT for patients, taking the IMT over time and tracking the IMT values during the follow-up studies. The method is also used for computing the IMT of the batch of patients in clinical databases automatically using the knowledge of ethnicity, demographics, age and gender and super controlled by the user.

19. The method as claimed in claim 1 wherein the method is can be run on a iPad, iPhone or running on a tablet using Android Operating System. The IMT-based implementation is using Java wrapper and the Graphical User Interface and can be used by a vascular surgeon or trained sonographers or vascular radiologist, neuroradiologist or even a cardiologist.
